(19) United States
(12) Patent Application Publication
(10) Pub. No.: US 2026/0243784 A1
(43) Pub. Date: Aug. 20, 2026

(54) SYSTEMS AND METHODS FOR AUTONOMOUS WORKFLOW MANAGEMENT

(71) Applicant: GRAIL, Inc., Menlo Park, CA (US)

(72) Inventors: David Jennions, Raleigh, NC (US); Derek Jenkins, Menlo Park, CA (US)

(73) Assignee: GRAIL, Inc., Menlo Park, CA (US)

(21) Appl. No.: 19/413,303

(22) Filed: Dec. 9, 2025

Related U.S. Application Data
(60) Provisional application No. 63/729,697, filed on Dec. 9, 2024.
(60) Provisional application No. 63/729,719, filed on Dec. 9, 2024.
(60) Provisional application No. 63/729,745, filed on Dec. 9, 2024.

(51) Int. Cl. G01N 35/00

(52) U.S. Cl. CPC G01N 35/00594; G01N 35/00732; G01N 2035/00633; G01N 2035/0094

(57) ABSTRACT

Systems and methods are disclosed, which may include: a hierarchical software architecture comprising a plurality of software layers that are ignorant of one another and are collectively configured to automate processing of at least one object through a workflow process, wherein the plurality of software layers comprise: a first software layer configured to guide the at least one object between one or more work cells during the workflow process; one or more second software layers, each being configured to manage execution of one or more steps associated with the workflow process in the one or more work cells; and one or more third software layers, each being configured to manage execution of tasks in furtherance of the one or more steps.

### CROSS-REFERENCE TO RELATED APPLICATIONS

This application claims the benefit of priority to U.S. Provisional Patent Application No. 63/729,697, filed on Dec. 9, 2024, U.S. Provisional Patent Application No. 63/729,719, filed on Dec. 9, 2024, and U.S. Provisional Patent Application No. 63/729,745, filed on Dec. 9, 2024, all of which are incorporated herein by reference in their entireties.

### TECHNICAL FIELD

The present disclosure relates generally to computer systems for automating a workflow and, more specifically, to systems and methods for dictating workflows to various sub-systems to autonomously achieve a specific task.

### BACKGROUND

Conventional laboratory setups, though often referred to as autonomous, typically require significant human intervention for critical steps, such as sample preparation and transportation between different machines and workstations, or regular interaction with a workstation to assist with fixing errors. This reliance on human involvement introduces variability and potential errors, thereby compromising the efficiency and accuracy of the overall process. Scaling these setups requires a larger workforce and increased space for redundant machines to maintain uptime, which is impractical and costly. Although there have been efforts to create fully automated laboratory workflows, these have faced significant challenges.

The background description provided herein is for the purpose of generally presenting the context of the disclosure. Unless otherwise indicated herein, the materials described in this section are not prior art to the claims in this application and are not admitted to be prior art, or suggestions of the prior art, by inclusion in this section.

### SUMMARY OF THE DISCLOSURE

According to certain aspects of the disclosure, systems and methods are described for providing a hierarchical software architecture for an autonomous laboratory workflow management system.

In some aspects, the disclosure is drawn to a computer system comprising a hierarchical software architecture. The hierarchical software architecture may include a plurality of software layers that are collectively configured to automate processing of at least one object through a workflow process. Each of the plurality of software layers may be configured to be ignorant to a functionality of another of the plurality of software layers. The plurality of software layers may include a first software layer of the plurality of software layers that is configured to guide the at least one object between one or more work cells during progression of the at least one object through the workflow process. The plurality of software layers may also include one or more second software layers of the plurality of software layers. Each of the one or more second software layers may be configured to manage execution of one or more steps associated with the workflow process in the one or more work cells. The plurality of software layers may further include one or more third software layers of the plurality of software layers. Each of the one or more third software layers may be configured to manage execution of tasks, performed by an instrument of a plurality of instruments contained in the one or more work cells, in furtherance of the one or more steps.

In another aspect, the disclosure is drawn to a computer system comprising a plurality of work cells, and each work cell may include a plurality of instruments. The computer system may also include a conveyance platform connecting at least a subset of the plurality of work cells. The computer system may also include one or more computer readable media storing instructions that are executable by the one or more processors to perform operations to identify a target work cell, from the plurality of work cells, that is configured to execute one or more subsequent steps in association with a workflow process; control, responsive to identifying the target work cell, a conveyance platform to transport one or more samples to the target work cell; and perform a series of processing steps on the plurality of samples within the target work cell, wherein a plurality of instruments within the target work cell are used to perform the series of processing steps. A first software layer may be configured to manage the identifying and controlling steps. A second software layer may be configured to manage the performing the series of processing steps within the target work cell. A third software layer may be configured to manage one or more of the plurality of instruments within the target work cell in furtherance of the one or more processing steps. Each of the first, second, and third software layers may be configured to be ignorant, and/or at least partially independent of, a functionality of the other software layers.

Additional objects and advantages of the disclosed embodiments will be set forth in part in the description that follows, and in part will be apparent from the description, or may be learned by practice of the disclosed embodiments. The objects and advantages of the disclosed embodiments will be realized and attained by means of the elements and combinations particularly pointed out in the appended claims.

It is to be understood that both the foregoing general description and the following detailed description are exemplary and explanatory only and are not restrictive of the disclosed embodiments, as claimed.

### BRIEF DESCRIPTION OF THE DRAWINGS

The accompanying drawings, which are incorporated in and constitute a part of this specification, illustrate several embodiments, and together with the description, serve to explain the principles of the disclosure.

FIG. 1 depicts a block diagram illustrating a hierarchical software architecture for executing the methods described herein, according to one or more embodiments of the present disclosure.

FIG. 2 depicts a diagram illustrating the input and output functions of a top software layer in the hierarchical software architecture, according to one or more embodiments of the present disclosure.

FIG. 3 depicts a diagram illustrating the input and output functions of a second software layer in the hierarchical software architecture, according to one or more embodiments of the present disclosure.

FIG. 4 depicts a diagram illustrating the input and output functions of a third software layer in the hierarchical software architecture, according to one or more embodiments of the present disclosure.

FIG. 5 depicts processing structures illustrative of the typical decisions that may be made and variations in processing introduced by the various layers of the hierarchical software architecture.

FIG. 6 depicts a diagram illustrating an exemplary logical layout of an automation system, according to one or more embodiments of the present disclosure.

FIG. 7 depicts a diagram illustrating the responsibilities managed by each software layer, and the communications between software layers, during execution of an exemplary process, according to one or more embodiments of the present disclosure.

FIG. 8 depicts an exemplary workflow that delineates a decision-making process for work cell assignment, according to one or more embodiments of the present disclosure.

FIG. 9 depicts an exemplary diagram designating the various processes, work cell types, and LIMS step names involved in a workflow process, according to one or more embodiments of the present disclosure.

FIG. 10 depicts an example computing system, according to one or more embodiments of the present disclosure.

### DETAILED DESCRIPTION OF EMBODIMENTS

The terminology used below may be interpreted in its broadest reasonable manner, even though it is being used in conjunction with a detailed description of certain specific examples of the present disclosure. Indeed, certain terms may even be emphasized below; however, any terminology intended to be interpreted in any restricted manner will be overtly and specifically defined as such in this Detailed Description section. Both the foregoing general description and the following detailed description are exemplary and explanatory only and are not restrictive of the features, as claimed.

Laboratory workflows, especially those involved in the processing and analysis of biological samples, require high precision and efficiency. Conventional laboratory setups, while often labeled as autonomous, are typically only semi-autonomous. More particularly, critical steps such as sample preparation, transportation between machines, initiating specific processes, and attending to system errors still depend heavily on human intervention. This dependency introduces variability and potential errors, adversely impacting the efficiency and accuracy of the workflows. Moreover, these systems are difficult to scale without a proportionate increase in workforce and space requirements, making it challenging to meet higher throughput demands. There is a need for a truly autonomous system that can handle the variability and complexity of laboratory workflows from start to finish with little, if any, human involvement.

Conventional efforts to automate laboratory workflows have primarily focused on either automating discrete steps of the overall process or fully centralizing control within a single system. With respect to the former, step-specific automation may involve automating individual steps of the laboratory workflow rather than the entire process. More particularly, certain machines in the overall workflow may be configured to perform specific tasks, such as sample preparation, analysis, or data collection. Examples of step-specific automation may include liquid handling robots that automate pipetting and reagent dispensing, automated PCR systems that perform polymerase chain reactions with minimal human intervention, and robotic sample storage systems that automatically store and retrieve samples from refrigerated units.

However, the advantages provided by these step-specific automation processes do not address the need for a seamless, end-to-end workflow. More particularly, human operators are still required to transport samples between machines, initiate processes, and handle exceptions. This type of fragmented automation introduces inconsistencies and potential errors, compromising the overall efficiency and accuracy of the workflow. Additionally, the throughput of step-specific automation systems is constrained by the need for manual handling between steps, and scaling these systems requires proportionally increasing the number of human operators. Furthermore, maintenance and downtime are additional issues, as individual machines require regular maintenance and calibration, leading to disruptions in the workflow and reduced productivity or redundancy of said individual machines can be added at great additional cost and physical space.

With respect to the latter of the two conventional solutions, centralized automation systems are designed to manage all aspects of the laboratory workflow through a single, overarching controller. These systems integrate various automated machines and instruments to perform specific tasks within the workflow. Some components of centralized systems may include robotics arms for moving samples between different workstations or components of workstations, automated liquid handling systems for tasks such as pipetting and reagent addition, and integrated software platforms that provide a unified interface for controlling all automated components.

Despite their promise, centralized automation systems face significant issues. For instance, one of the primary problems is computational overload. More particularly, centralized systems require a single controller to manage numerous variables, including machine status, sample location, and timing for each process step, to name a few. Attempts to harmoniously manage these variables has conventionally led to computational overload, thereby causing delays, inefficiencies, and frequent system failures. Additionally, these systems still depend heavily on human operators to prepare samples, initiate processes, and troubleshoot issues. This reliance on human intervention introduces variability and potential errors, reducing overall efficiency and accuracy.

Scalability is another challenge encountered by centralized systems. Specifically, scaling these systems to handle higher throughput (e.g., population-level sample analysis) involves adding more automated machines, which increases the use of and reliance on the central controller, and results in computational overload. This scaling approach is limited by the controller's ability to manage additional components and the increased space required for additional machinery. Integration and compatibility issues also arise when integrating automated components from different vendors, making seamless communication and coordination between diverse machines and instruments complex and prone to errors. Lastly, centralized systems typically require complete shutdowns to conduct maintenance, upgrades, or repairs, leading to significant workflow disruptions and productivity loss.

Additionally to the foregoing, some industries, such as the automotive sector, have successfully implemented end-to-end automation, benefitting from the standardized and repetitive nature of their processes. In these industries, automation systems are designed to handle a relatively narrow range of tasks with consistent inputs and outputs, allowing for efficient and predictable operations. In contrast, laboratory workflows present a unique challenge due to their inherent variability. More particularly, laboratories often deal with a wide range of sample characteristics, sample quantities, sample types, processing methods, and analytical requirements, making it difficult to standardize workflows in the same way as the manufacturing industries. This variability introduces complexity in automation, as systems must be flexible enough to adapt to different protocols and procedures or respond to variations in a given sample including its reactions to the components of an assay. As a result, fully automating laboratory workflows requires advanced systems capable of dynamic decision-making and adaptability, which are often more challenging to develop and implement than the relatively straightforward automation systems used in industries with less variability.

Accordingly, the novel concepts described herein are generally directed to a novel computer system designed to achieve complete autonomy in laboratory workflows by leveraging a hierarchical software architecture, in which each layer of software in the hierarchical architecture is purposefully left generally ignorant or agnostic of the other layers.

This novel system includes multiple independent software layers that distribute computational tasks at different layers of abstraction, thereby reducing data complexity and preventing computational overload. At the highest level, the top software layer orchestrates the overall workflow, managing the flow of samples and materials between various work cells based on context data such as assay protocols, system component status, and inventory levels. The top software layer operates without awareness of the details of operations in the various work cells. The second software layer, functioning as a scheduler, manages tasks within individual work cells by mapping required work to available resources, allowing for the simultaneous processing of multiple sample batches. The second software layer operates without awareness of the other work cells or the broader workflow context. The third software layer controls specific instruments within the work cells, executing discrete operations without awareness of the other instruments in the work cell or the broader workflow context.

The innovative architecture described above ensures that each software layer operates independently and agnostically, enhancing the system's reliability and scalability. Additionally, the system's modular design allows for the addition or removal of hardware components, such as work cells and instruments, without necessitating system-wide shutdowns, thus maximizing uptime and operational efficiency. Moreover, the novel system may include an integrated information repository, which facilitates communication between layers, storing progress and quality assurance data and enabling the top software layer to monitor the overall health and workflow status of the system. Accordingly, by eliminating the need for human intervention in work allocation, sample handling, and movement, this fully autonomous system reduces the risk of human error, thereby enhancing efficiency and enabling the processing of large numbers of samples (e.g., millions of samples) with precision and speed.

A system that incorporates the innovative concepts described herein may be well suited, for example, for sample preparation and analysis in cancer detection analysis, where precision and consistency are important considerations. For instance, the system may be capable of efficiently processing a large quantity of samples or different types of biological samples, such as liquid, (e.g., blood, urine, saliva, stool, cerebrospinal fluid, pleural fluid, interstitial fluid, etc.), tissue, or biopsy specimens, to extract nucleic acids and other biomarkers important for cancer diagnosis. The system's hierarchical software architecture may be configured to manage these processes, which may not only increase the efficiency of the sample preparation process but also reduce the potential for human error, thereby enhancing the reliability and accuracy of downstream cancer detection assays, while maintaining or even improving long-term costs.

The concepts described herein introduce several significant technical improvements. For instance, by dividing tasks among multiple independent software layers and purposefully keeping the software layers agnostic to one another, the system reduces the computational burden on any single layer. Each layer is responsible for specific functions, allowing for more efficient processing and avoiding the risk of system overload. This distribution enhances the system's reliability and performance, preventing the computational bottlenecks that commonly plague centralized systems.

Additionally, the system's architecture supports modifications, repairs, and updates while remaining operational. This “online” maintenance capability promotes continuous operation and minimal disruption, a significant improvement over conventional systems that require complete shutdowns for even routine maintenance tasks. In an aspect, dynamic task reallocation allows the top software layer to dynamically reassign work to alternative work cells or instruments if a specific component needs to be temporarily taken offline for servicing. Furthermore, the modular design of the novel system allows it to easily scale by the addition or removal of work cells and instruments as needed, without significantly compromising performance. This scalability is achieved without the need for extensive reconfiguration or system downtime, making it possible to adapt to varying throughput requirements and evolving laboratory needs. The ability to integrate additional hardware components ensures that the system can grow in response to increasing demand or new technological developments or shrink in response to decreased throughput needs or upstream efficiencies. Furthermore, the system is configured to autonomously manage complex workflows, including the movement and processing of samples, with precision and speed. This autonomy enables the system to process millions of samples accurately, improving throughput and reliability.

The subject matter of the present disclosure will now be described more fully hereinafter with reference to the accompanying drawings, which form a part hereof, and which show, by way of illustration, specific exemplary embodiments. An embodiment or implementation described herein as “exemplary” is not to be construed as preferred or advantageous, for example, over other embodiments or implementations; rather, it is intended to reflect or indicate that the embodiment(s) is/are “example” embodiment(s). Subject matter may be embodied in a variety of different forms and, therefore, covered or claimed subject matter is intended to be construed as not being limited to any exemplary embodiments set forth herein; exemplary embodiments are provided merely to be illustrative. Likewise, a reasonably broad scope for claimed or covered subject matter is intended. Among other things, for example, subject matter may be embodied as methods, devices, components, or systems. Accordingly, embodiments may, for example, take the form of hardware, software, firmware, or any combination thereof. The following detailed description is, therefore, not intended to be taken in a limiting sense.

Throughout the specification and claims, terms may have nuanced meanings suggested or implied in context beyond an explicitly stated meaning. Likewise, the phrase “in one embodiment” or “in some embodiments,” or “in one aspect” or “in some aspects” as used herein does not necessarily refer to the same embodiment or aspect, and the phrase “in another embodiment” or “in another aspect” as used herein does not necessarily refer to a different embodiment or aspect. It is intended, for example, that claimed subject matter include combinations of exemplary embodiments in whole or in part.

Although the present disclosure is primarily described in the context of biological sample processing, it should be understood that the system architecture is not limited to this application and may be adapted for a wide variety of other fields and tasks that require precise, multi-step processing. For instance, this system may be effectively employed in the manufacturing industry for the assembly of electronic components, where different work cells may be configured to handle tasks such as soldering, component placement, and quality control. Similarly, the concepts described herein may be utilized in the pharmaceutical industry for drug formulation and packaging, ensuring that each step, from ingredient mixing to final packaging, is carried out accurately and efficiently without human intervention.

The computer system described herein may be designed to handle a wide variety of sample types, making it suitable for diverse applications across multiple industries. The system's modular and flexible architecture allows it to efficiently process samples with different properties and requirements. For instance, non-limiting exemplary types of samples that may be processed through the disclosed system include: biological samples (e.g., tissue samples, blood samples, urine samples, saliva samples, fecal samples, or other fluid samples, etc.), environmental samples (e.g., water samples, soil samples, etc.), food and beverage samples, forensic samples (e.g., crime scene samples, toxicology samples, etc.), industrial samples (e.g., pharmaceutical samples, chemical samples, etc.), and the like. In an aspect, the versatility of the system may allow it to be adapted for specific sample types and processing requirements. Work cells within the system may be configured with specialized equipment and reagents tailored to the unique properties of each sample type, ensuring accurate and efficient processing from start to finish. This capability makes the system a valuable tool across various fields, enhancing the reliability and throughput of analytical workflows.

The following definitions clarify key terms used throughout this disclosure to describe the various aspects of the autonomous workflow management system.

As used herein, a “hierarchical software architecture” may refer to a layered structure of the software system, where each layer operates independently and performs specific functions within the overall workflow. For instance, this architecture may include a top software layer, a second software layer, and a third software layer, each managing different aspects of the processing workflow. Although three software layers are used in descriptions and many examples herein, more or fewer layers may be used in certain embodiments based on the complexity of the software system and individual tasks thereof.

A “workflow” may refer to the high-level ordered set of work necessary to complete an overall goal. It encompasses the sequence of stages that need to be executed to achieve the desired end result. In the context of this disclosure, a workflow dictates the progression of sample processing from one stage to the next, outlining the major phases required to process and analyze biological samples. For instance, given a DNA extraction and analysis scenario, the workflow may designate the following sequence of stages: sample collection, sample preparation, DNA extraction, DNA purification, DNA quantification, sequencing preparation, sequencing, and data analysis.

A “recipe” may refer to the set of parameters that define how a workflow should actually be executed. These parameters may include specific instructions, conditions, and/or settings that tailor the execution of a workflow to meet the particular requirements of different products or samples. Recipes may vary significantly between different types of samples processed, products, assays, or desired outputs, providing the detailed operational guidelines that direct how each step in the workflow is carried out. In essence, the recipe adds the specificity and customization to the more generalized framework of a workflow. For instance, given the workflow for a DNA extraction and analysis scenario, as previously described above, the recipe for executing the workflow for a set of blood samples may include: preparation of the samples by centrifuging the blood at 1500 g for 10 minutes to separate plasma and utilize a specific lysis buffer for lysing cells, using a specified enzyme or chemical reaction to prepare the DNA for epigenomic analysis, extracting DNA utilizing a silica-based extraction kit and incubating the samples with proteinase K at a specific temperature for a specified time, purifying the DNA using an ethanol wash and eluting the DNA in a specified volume of elution buffer, performing DNA quantification using a spectrophotometer to measure DNA concentration, performing sequencing preparation via utilizing a specific library preparation kit for the sample type and performing PCR amplification (e.g., with a specified number of cycles), sequencing the samples using a predefined sequencing platform, and aligning sequences, performing variant calling, or other analyses using a specific software, model, or analytical pipeline. Should DNA need to be extracted from urine, saliva, or fecal samples instead of blood samples, a different recipe may be employed (e.g., different types of collection tubes, different centrifugation parameters, etc.) up to a certain point (e.g., once DNA is extracted). Although the specific example above is provided as an example of a recipe, it is understood that other recipes, or modifications to this example recipe, may be used. Embodiments of the specification are not limited to the particular recipe set forth above.

A “workflow process” combines the workflow and the recipe. More particularly, the workflow process may correspond to a predefined, structured set of instructions and procedures designed to guide the sequential processing of a sample through various stages of analysis or production. In the context of this application, the workflow process serves as a comprehensive roadmap that outlines each specific step required to process a sample from start to finish, ensuring that all necessary tasks are performed in the correct order, by the correct work cell, and under the appropriate conditions. In some aspects, the workflow process may not specify the exact software layers and/or instruments that may be used to process the sample. For example, if a work cell includes three centrifuges A, B, and C, the workflow process may simply indicate that one of the steps that is used to process the sample is centrifugation, and may not specify which particular centrifuge to use. In another example, if a work cell includes three centrifuges A, B, and C, the workflow process may simply indicate that the sample may be processed by only centrifuge A or B, because centrifuge C may be down or temporarily occupied or otherwise unavailable. A second software layer associated with a work cell may then use the information in the workflow process to decide which of centrifuges A and B is the best choice to process the sample, e.g., in order to improve the efficiency of sample processing.

A “Laboratory Information Management System (LIMS)” may refer to a data storage repository that records information related to sample processing. LIMS may be configured to store details such as sample status, workflow progress, and system help. Each software layer may publish execution data to LIMS, and the top software layer may periodically or continuously access this repository to make informed decisions and/or to modify the recipe and/or workflow process (e.g., for a given biological sample in processing).

A “work cell” may refer to a cluster of instruments, each designed to perform specific steps in the process. Work cells may operate semi-independently, contributing to the system's modularity and scalability. Different work cells may be configured for various stages, e.g., including DNA extraction, amplification, or sequencing preparation.

An “activity work cell” may correspond to a specialized unit within an autonomous workflow management system designed to execute specific workflow steps. Each activity work cell may contain a group of equipment and instruments necessary for processing steps according to predefined workflows. For instance, the equipment within an activity work cell may include one or more robotic components, centrifuges, pipetting instruments, PCR machines, etc. Each type of activity work cell may be primarily defined by the equipment involved, the input expected (e.g., sample in a specific state and/or reagents), and the anticipated output.

A “buffer work cell” may correspond to a specialized storage unit within the autonomous workflow management system that is designed to store materials, reagents, and intermediate products for on-demand usage by activity work cells. Buffer work cells do not execute workflow steps but facilitate smooth transitions and continuous operations by ensuring that materials are readily available. Buffer work cells may include equipment such as refrigerators, freezers, climate control systems (e.g., humidifiers, dehumidifiers), ambient storage units, and automated retrieval systems (e.g., robotic arms, conveyance systems, etc.). In some aspects, buffer work cells may also include multi-temperature storage compartments to maintain the stability and integrity of stored materials, ranging from ultra-low temperatures to ambient conditions.

A “conveyance platform” may refer to one or more mechanisms that connect work cells and facilitate the movement of samples between them. The conveyance platform may ensure the transfer of samples and assay materials (e.g., reagents and disposables), allowing the system to maintain continuous operation. Exemplary conveyance platforms may include one or more hardware components, including pucks, tracks, belts, grippers, etc. A conveyance platform may be centralized (e.g., comprise one or more large-scale installations connecting many work cells along a defined path) or decentralized (e.g., comprise many components capable of independent movement, such as an autonomous transport device capable of connecting two end points without a defined path or order). In some instances, a conveyance platform may include centralized or decentralized components.

A “robotic component” may be a component in the work cell that is designed to perform a variety of precise and repetitive tasks that are important for processing samples. As used herein, the robotic component may correspond to, e.g., a “robotic arm.” The robotic arm is a highly versatile, mechanical device that may be equipped with multiple joints and degrees of freedom, which may mimic the dexterity and range of motion of a human arm, enabling it to execute complex maneuvers with high precision and accuracy. This may include tasks such as pipetting exact volumes of liquid, transferring samples between different containers or instruments, and/or from the conveyance platform to different containers or instruments, mixing reagents, and positioning samples within various instruments. In the context of this application, a robotic component may refer to a single robotic component or a series of robotic components.

For the purposes of this disclosure, the terms “stage,” “step,” “task,” and “operation” have specific definitions and are not synonymous.

A “stage” may refer to a broader portion of the assay workflow that encompasses a series of related processes aimed at achieving a specific intermediate goal. Stages represent major phases within the overall workflow, such as DNA pre-extraction, extraction, and enrichment. For example, the pre-extraction stage may involve initial sample preparation and handling, the extraction stage may involve isolating target molecules, and the enrichment stage may involve enhancing the concentration and purity of the extracted molecules. The top layer of software may coordinate which stage a sample moves to next. In some instances, a single stage may be split between two work cells to optimize resource utilization and enhance processing efficiency. This division may, if utilized, allow the system to balance workloads by distributing actions based on the availability and capability of each work cell.

A “step” may refer to a discrete process within a stage. Steps are specific actions that need to be performed to progress through a stage, each with defined inputs and outputs. The second software layer may coordinate these steps, mapping required tasks to available resources within a work cell. For example, within the extraction stage, steps may include digestion, extraction consolidation, and washing and elution.

A “task” may correspond to a specific unit of work within a step, often corresponding to a single function performed by an instrument or set of closely related functions. For instance, to execute the step of digestion, an instrument may perform a series of tasks including centrifugation, peeling, internal storage, plasma transfer, mixing, heating, incubation, etc. Each of these tasks is a distinct unit of work that can be assigned to a specific instrument or piece of equipment designed to perform that function.

An “operation” may correspond to the most granular level of activity, representing a logical, distinct part of a task, typically involving a single motor control or a specific action performed by the instrument. More particularly, operations may be thought of as the basic actions that, when combined, accomplish a task. For instance, in the task of adding a reagent to a sample tube, there may be an operation to draw the reagent (e.g., a motor is controlled to raise the plunger, creating a vacuum that draws the reagent up into the pipette tip) and another operation to dispense the reagent (e.g., the motor may be controlled to lower the plunger, pushing the reagent out of the pipette tip and into the sample tube).

A “sample” may refer to an individual sample or a batch of samples, for convenience, unless specified otherwise. Samples may be stored in sample tubes (e.g., individually or pooled) or may be stored in devices configured to hold and separate multiple samples (e.g., multi-tube racks, wells of multiwell plates) to enable simultaneous manipulation and treatment of multiple samples.

A “memory device” may be any suitable device that can store electronic data. A suitable memory device may contain a computer readable medium that stores instructions that can be executed by a processor to implement a desired method. Examples of memory devices may contain one or more memory chips, disk drives, etc. Such memory devices may operate using any suitable electrical, optical, and/or magnetic mode of operation.

A “processor” may refer to any suitable data computation device or devices. A processor may include one or more microprocessors working together to accomplish a desired function.

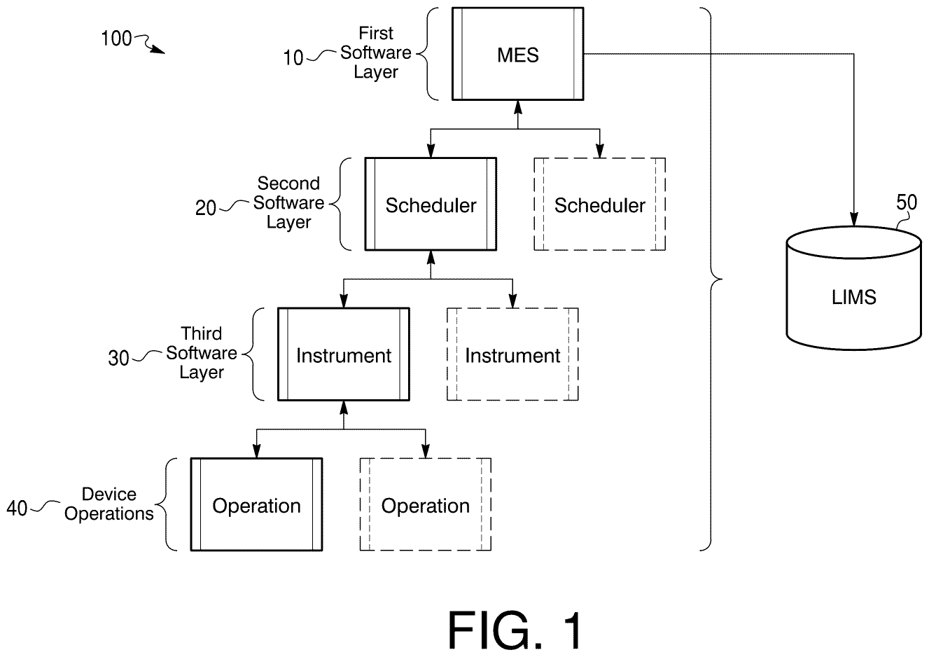

FIG. 1 depicts a block diagram illustrating a hierarchical software architecture 100 for a fully automated system, according to embodiments of the disclosure. The software architecture 100 may contain a plurality, e.g., three, software layers, including the top or first software layer 10, the second software layer 20, and the third software layer 30, with each layer responsible for its own specific set of data. The third software layer 30, which corresponds to instrument control within a work cell, may be responsible for executing specific device operations 40, as further discussed herein. In an aspect, the three software layers 10, 20, 30 may be in the form of software components that are stored on or in a memory and/or computer readable medium and that work with one or more processors (e.g., data processors) residing on one or more computer apparatuses. For example, all three layers 10, 20, 30 may reside on or in a computer readable medium on one computational apparatus with one or more processors (e.g., microprocessors). Alternatively, the three layers 10, 20, 30 may reside on or in three computer readable media residing on three separate computational apparatuses, each with one or more processors (e.g., microprocessors). In some aspects, the top software layer 10 may reside in a first computational apparatus (e.g., a first server computer, virtual machine, or thread), the second software layer 20 may reside in a second computational apparatus (e.g., a second server computer, virtual machine, or thread), and the third software layer 30 may reside in a third computational apparatus (e.g., a third server computer, virtual machine, or thread). Alternatively, a subset of the three layers 10, 20, 30 may reside on or in two computer readable media residing on two separate computational apparatuses, each with one or more processors (e.g., microprocessors), such that two software layers reside on or in one computer readable media and one software layer resides on its own computer readable media.

The Laboratory Information Management System (LIMS) 50 is a component within the hierarchical software architecture of system 100. Serving as a centralized information repository, LIMS 50 stores data generated throughout the workflow process. More particularly, LIMS 50 functions to provide a comprehensive database where relevant information about samples, processes, and system status is recorded. This includes data on sample identity, processing steps and results, reagent usage, and the status of various system components. By maintaining a centralized repository of such information, LIMS 50 enables the system to track the progress of each sample in real time, ensuring that all actions are accurately documented and traceable. This traceability promotes the integrity of the workflow and compliance with regulatory standards. In an aspect, LIMS 50 interfaces with each of the three software layers 10, 20, 30, facilitating the flow of information across the system 100. One or more layers of software 10, 20, 30 may publish information to LIMS 50 while interfacing with samples or while awaiting samples. As further discussed herein, the top software layer 10 may access LIMS 50 to obtain context data, such as inventory levels, sample positions in the workflow process, instrument and component health indications, and the like, which are necessary for high-level decision-making and workflow orchestration. It is important to note that although FIG. 1 and the balance of this disclosure discusses a software architecture that contains three software layers, such a designation is not limiting. Specifically, fewer or additional software layers may be utilized in a system to facilitate the concepts described herein. For instance, instruments within the system may also be configured to oversee specific sub processes independently. This capability may allow individual instruments to manage tasks such as calibration, quality control checks, and data validation.

The fully automated system for which the hierarchical software architecture 100 is constructed may include a variety of different hardware components. Provided below are descriptions of hardware components that may be utilized in the fully automated system. However, it is important to note that the number and type of hardware components may vary between systems and may be tailored to the overall goal of the fully automated system. Additional detail regarding how these hardware components interact and/or are controlled by the various system layers 10, 20, 30 are further provided herein.

The fully automated system may include one or more work cells which, in general, refer to a cluster of instruments that are each designed to execute various tasks in the furtherance of specific steps in the process workflow. More particularly, each work cell may be equipped with a variety of instruments and components tailored to the specific sample processing step(s) it was organized to perform. Some or all work cells may include a robotic component, such as a robotic arm, that may be linked to and controlled by a second software layer responsible for coordinating and executing tasks within the work cell. For instance, as an example, a pre-amplification processing work cell may be designed and equipped to handle various preparatory actions required for biological sample processing. It may contain a robotic arm capable of performing actions such as pipetting, mixing, and/or transferring samples between different instruments. This robotic arm may ensure that each step, task, and/or operation is repeatedly executed accurately, reducing the risk of human error. In an aspect, the work cell may additionally be equipped with other instruments, such as an automated pipettor (e.g., that facilitates precise liquid handling, allowing for the accurate addition of reagents or samples into plates or tubes), a centrifuge (e.g., which is utilized to separate components of the sample based on density), a heater/shaker (e.g., which may provide the necessary heating and agitation to ensure proper mixing and reaction conditions during various processes), an ID scanner, such as a one-dimensional or two-dimensional barcode scanner, (e.g., which may be utilized to ensure accurate identification and data logging), and the like. As another example, another type of work cell may be a post-amplification processing work cell, which may be designed to handle tasks related to the amplification and subsequent processing of biological samples. Such a work cell may include a variety of equipment, some or all of which may be different from the pre-amplification work cell, including: a polymerase chain reaction (PCR) machine (e.g., which is utilized for amplifying DNA samples through PCR, thereby increasing the quantity of DNA), a thermocycler (e.g., which may be utilized to facilitate the cycling of temperatures necessary for the denaturation, annealing, and extension phases of PCR), an automated gel electrophoresis system (e.g., which may be utilized for separating and analyzing DNA fragments, providing a means to verify the success and quality of the amplification process), and/or other devices not explicitly listed here.

Additionally to the foregoing, buffer work cells may be included in the system and may serve as intermediary storage units that help regulate the flow of samples and materials between different stages of processing. More particularly, unlike the activity work cells described above, these specialized units may not perform active processing steps or tasks.

Instead, they may be configured to act as temporary holding areas for samples, reagents, and other consumables (e.g., items that may be used up or consumed during laboratory processes, such as reagents and chemicals, plastics and glassware, filters, membranes, laboratory gases, etc.). By providing a buffer between active work cells and the conveyance platform, these buffer work cells help to smooth out the fluctuations in processing rates, ensure smooth transition from the conveyance platform to an activity work cell, and manage unexpected delays, thereby allowing subsequent stages of the workflow process to proceed without interruption. In an aspect, buffer work cells may include one or more storage environments, such as one or more of ambient, refrigerated, and/or freezer storage environments, to maintain the integrity of samples and reagents. Additionally, in some aspects, they may enable the system to balance workloads by temporarily storing batches of samples when downstream work cells are occupied or during peak processing times, or may allow consumables to be delivered to work cells at times when samples are not being actively conveyed along a route to reduce sample down time and avoid unnecessary traffic on the conveyance platform. In particular, because a robotic arm may be required to place reagents, consumables, and other items, the addition of a buffer work cell with an accompanying robotic arm upstream of an activity work cell ensures that the activity work cell is not slowed down by actions involving the repositioning of materials that are not presently required for sample processing.

Some or all of the work cells (e.g., including both activity work cells and buffer work cells) may further include various types of multi-temperature storage, which are designed to provide optimal storage conditions for a wide variety of biological samples and reagents. These storage units may be capable of maintaining and regulating multiple temperature zones, ranging from ultra-low temperatures of −80° C. to standard refrigeration temperatures of 4° C., and ambient room temperature. This capability allows different types of biological materials, each with its unique stability and storage requirements, to be preserved under ideal conditions. For instance, nucleic acids like DNA and RNA, which are often sensitive to degradation, may be stored at −80° C. to maintain their integrity over extended periods or may be stored at −20° C. over shorter periods. Meanwhile, enzymes and other reagents that require cold storage, but not ultra-low temperatures, may be kept at −20° C. or 4° C., so that they remain active and effective. The multi-temperature storage units may be integrated, with multiple temperature zones in a single standalone unit, or may be modular, with different temperature zones handled by separate standalone units, to allow for expansion based on the storage needs of a given work cell. In an aspect, the modular design of the multi-temperature storage units allows them to be easily integrated into existing laboratory setups and scaled according to the needs of the laboratory. Additionally, they may be configured to accommodate a wide range of sample types, from relatively “whole” samples (e.g., blood, urine, saliva, fecal samples) to components thereof, to purified nucleic acids and reaction mixtures.

In an aspect, access to the multi-temperature storage units may be facilitated by a robotic component integrated into the work cell. These robotic components may be equipped with control mechanisms and sensors, enabling them to navigate within the storage units. Additionally or alternatively, the multi-temperature storage units may be communicatively coupled with the system 100 such that requests to make available particular samples or materials are received from the appropriate layer of the hierarchical software architecture 100 and the robotic components may be equipped with mechanisms and programming to retrieve the requested samples and materials as presented. When a sample or reagent is needed, the robotic component may receive instructions from the second software layer, which may identify the location of the required item within the storage unit. The robotic arm may then move to the designated storage compartment, e.g., using its sensors to accurately position itself and retrieve the sample or reagent without compromising its integrity. In an aspect, the robotic arms may be designed to handle the diverse environmental conditions within the multi-temperature storage units. For instance, when accessing samples stored at −80° C., the robotic arm may be capable of operating in ultra-low temperatures, ensuring rapid and reliable retrieval. Similarly, the robotic arm may be capable of operating with items stored at −4° C. or room temperature. While large temperature differences between storage units are described in this example, it is considered that at least in some work cells, the temperatures of storage units within a work cell may not vary as widely, or at all. In an aspect, the robotic arm may adjust its handling techniques to accommodate different storage conditions. In an aspect, some or all of the multi-temperature storage units may be equipped with automated doors or hatches that the robotic arm can open and close (or that may be configured to automatically open or close in response to sensing that the robotic arm is approaching or in response to receiving requests for samples or materials stored therein). These doors may be synchronized with the robotic arm's movements to minimize the exposure of stored items to external environmental conditions, thereby preserving their stability.

In an aspect, inventory tracking and management within the laboratory system may be used for maintaining the efficiency and reliability of the system as a whole. This may be achieved through a combination of various technologies and systematic processes designed to promote control over the storage and retrieval of biological samples, reagents, and consumables. In an aspect, each sample or reagent stored within the multi-temperature storage units, buffer work cell, and laboratory system generally, may be assigned a unique identifier, e.g., in the form of a barcode, QR code, or RFID tag. These identifiers may be scanned and recorded (e.g., to LIMS) at the time of storage, thereby logging details such as one or more of the sample type, patient identifier associated with the sample, sample batch identifier, age of the sample, storage temperature, quantity, and location within the storage unit. In an aspect, the status of items within the storage units may be monitored, and updates may be recorded in substantially real-time. For instance, when a sample or reagent is placed into a storage compartment, the robotic arm may scan its identifier and record the storage conditions and location to LIMS. As further described herein, LIMS may be queried by the top software layer to determine the availability and status of specific samples or reagents at any given time. In an aspect, as samples and reagents are retrieved for use, the samples and reagents may be scanned again, updating the inventory status in LIMS to reflect the removal of the item from storage. If a sample is returned to storage after partial use, its identifier may be scanned once more, and the updated information, such as remaining quantity and new storage location, may be recorded in LIMS. This dynamic tracking capability ensures that the inventory records are accurate and up-to-date.

In an aspect, the work cells may be situated along a conveyance platform, which connects work cells and facilitates the movement of samples throughout the processing pipeline. The conveyance platform may be controlled by the top software layer, which ensures that samples are transported efficiently and accurately between different stages of the workflow. In an aspect, the conveyance platform may take a variety of different forms. For instance, the conveyance platform described throughout this disclosure is a centralized motion platform that includes a conveyor belt or magnetic track that is configured to support and transport samples between different work cells. For example, the conveyance platform may be equipped with pucks, or other carriers, which are moved along the belt or track and are designed to transport a variety of sample containers (e.g., well plates, petri dishes, tubes, other laboratory vessels or specialized carriers depending on the nature of the sample and the requirements of the specific workflow, etc.) or other materials. These pucks may be configured to maintain the stability of the samples during transit, inhibiting spills, contamination, or mishandling. In an aspect, the design of the pucks may be customized to accommodate different types and sizes of samples, ensuring compatibility with the diverse needs of laboratory processes.

Beyond conveyor belts, other potential types of conveyance platforms may be utilized in lieu of, or in addition to, conveyor belts that are employed based on the specific requirements of the workflow and laboratory setup. For instance, one alternative is a robotic system, which may pick up and relocate samples with high precision. Autonomous robots or robotic arms may navigate paths and handle samples with care. In another aspect, a track-based shuttle system may be utilized, where autonomous shuttles move along fixed tracks to transport samples. These shuttles may be programmed to follow specific routes, stopping at designated work cells to deliver and pick up samples. The flexibility of this system may allow for dynamic routing and efficient management of multiple samples simultaneously. In yet another aspect, a pneumatic tube system may be leveraged to transport small, sealed containers quickly over longer distances within a facility. This system may utilize compressed air to propel tubes through a network of pipes, delivering samples rapidly and reliability to various destinations. In another aspect, a drone system may be used to transport carriers, samples, or materials between, e.g., a central storage unit and the work cells. Suitable systems may be, e.g., free-standing, or may be mounted to one or more of the floor, ceiling, or wall. In an aspect, each of the foregoing types of conveyance systems, or others, may be selected and utilized based on factors that are appropriate for the type of workflow, e.g., the layout of the laboratory, the nature of the samples being processed, and/or the specific workflow requirements. In an aspect, each of the foregoing types of conveyance systems may be implemented alone or, alternatively, may be used in combination if appropriate.

In an aspect, the system may contain a central repository that serves as the primary storage and staging area for samples and reagents before they are conveyed by the conveyance platform to work cells and processed. Upon arrival in the central repository, samples may be accessioned, cataloged, and stored in the central repository, which may be equipped with one or more temperature-controlled storage units capable of preserving a plurality of samples at optimal conditions. In an aspect, when a sample is scheduled for processing, it may be retrieved (e.g., using one or more robotic components) and transported by the conveyance platform from the central repository to a designated work cell (e.g., the first work cell in the workflow). In other aspects, the central repository may serve as long term storage for reagents and reserve amounts of samples after primary processing is complete. Samples may be accessioned and processed immediately after receipt at the library.

Referring back to FIG. 1, the top or first software layer 10, also known as the “Manufacturing Execution System (MES)” or “orchestrator,” serves as the central command unit of the entire system. This layer is primarily focused on managing the overall flow of samples and materials between different work cells, ensuring a seamless and efficient process from start to finish. It acts as the high-level decision-maker, utilizing comprehensive context data such as assay protocols, timing requirements, system component status, and inventory levels to make informed decisions about sample handling and routing. In an aspect, the MES may be configured to ensure that all necessary materials and instructions are present for the lower-level execution layers to function without interruption. By doing so, it maintains a steady and controlled flow of samples through the system, mitigating bottlenecks and optimizing throughput. This layer may be configured to handle hundreds, thousands, or millions of individual samples throughout the process, being apprised of their progress and dynamically adjusting the workflow as needed to accommodate the varying workloads, system status changes, and unexpected disruptions.

Moreover, the MES may be configured to operate with a high degree of autonomy, thereby reducing the need for human intervention in sample management. In an aspect, the MES may coordinate the deployment of samples to multiple work cells, enabling them to process different samples simultaneously. For instance, while one work cell may be engaged in DNA extraction, another may be performing PCR amplification, and yet another may be conducting sample analysis.

It is important to note that the MES does not exert control over the specific processing steps occurring within each work cell and the tasks and operations performed by each instrument, but rather, coordinates the flow of samples and materials among work cells to ensure that each work cell timely receives the samples and resources it needs to perform its designated procedures. The MES knows what each work cell does (i.e., what the input and the output of each work cell will be), but not the individual steps performed within the work cell in order to produce the output. More particularly, the MES may be designed with a purposeful ignorance of the specific functionalities of the lower software layers. This architectural choice is integral to the system's efficiency, scalability, and reliability. More particularly, by not delving into the detailed operations of the subordinate layers, the MES can focus on high-level decision-making and workflow management without being bogged down by the complexities of individual step, task, and/or operation execution. Accordingly, although the MES layer may be configured to manage the overall flow of samples and materials through the system, it does not need to know the specific methodologies employed by the lower layers to execute these procedures.

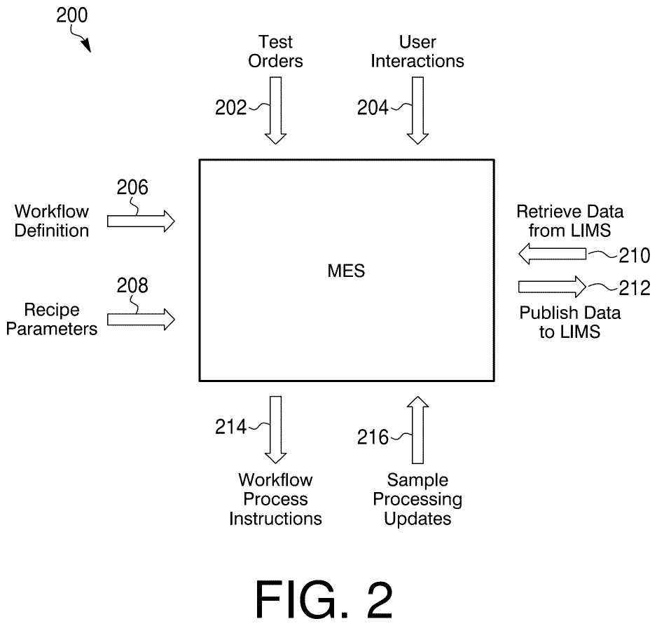

To effectively manage the flow of samples and materials throughout the system, the MES may reconcile a variety of different types of data. For instance, referring now to FIG. 2, diagram 200 illustrates the various types of inputs and outputs that are received and transmitted by the MES.

The MES may receive test orders 202, which are formal requests or directives to perform specific sample preparation, tests, or assays on a set of biological samples. These test orders 202 may originate from external sources such as clinicians, researchers, or other laboratory users who require specific analyses to be conducted on samples they submit. The components of a test order 202 may include one or more of: sample information (e.g., sample ID (a unique identifier for each sample)), sample type (blood, urine, tissue, etc.), quantity/volume (amount of sample provided, etc.), requested tests (e.g., test type (specific sample preparation, test, or assay to be performed)), priority level (urgency of test such as routine or urgent), special instructions (any special handling or preparation instructions required, etc.), client information (e.g., client ID (identifier for the person or organization requesting the test)), contact information (details for communication and reporting results, etc.), and/or submission date and deadline (e.g., date of submission (when the test order was placed), required completion date (when the results are needed), etc.). Test orders 202 may be used by the MES to determine and, in some instances, to prioritize the workflows that need to be executed. More particularly, the MES may take the information from these orders and translate them into actionable workflow process instructions.

The MES may be subject to various interactions 204 from users that may be provided to manage, monitor, and adjust laboratory workflows. These interactions may be important for ensuring that the system operates efficiently and effectively, even in an autonomous environment. Users, who may include laboratory technicians, researchers, quality control personnel, etc., may interact with the MES through a user interface that allows them to input data, receive updates, and make adjustments to ongoing processes. In an aspect, users may input various types of data into MES, such as new test or processing orders, sample details, reagent batch information, etc. Additionally or alternatively, users may manually adjust workflow parameters based on the characteristics of a sample batch and/or in response to receipt of real-time processing data. For example, users may change the temperature settings for an incubation step or modify the duration of a centrifugation process to optimize results. Users may also manually modify the work cells available for processing samples such as to temporarily disable work cells so that maintenance, repairs, or restocking may be performed. Additionally or alternatively, in cases where automated decisions need human intervention, users may override the system's actions, which may be necessary during unexpected events or when special handling of samples is required. Other types of user interactions with the MES, not explicitly discussed here, may also be provided.

The MES may utilize the workflow definition 206 and recipe parameters 208 to construct the workflow process, which may define the set of instructions and procedures designed to guide the processing of a sample or a batch of samples through various stages of analysis or production. With respect to the former, the workflow definition 206 refers to the high-level description and organization of the sequence of steps required to complete a specific laboratory process or achieve an overall goal. For instance, the workflow definition 206 may delineate that in a DNA extraction portion of the workflow process, the steps may include sample lysis, DNA binding, washing, and elution. In an aspect, the workflow definition 206 may also specify the dependencies between steps, indicating which steps must be completed before others can begin. This ensures that the workflow proceeds logically and that prerequisite conditions are met.

With respect to the latter, recipe parameters 208 refer to the specific instructions, conditions, and settings that define how a workflow should be executed. More particularly, these parameters may provide the detailed operational guidelines necessary for tailoring the execution of each step in the workflow to meet the particular requirement of different products, samples, or experimental conditions. Accordingly, while the workflow definition 206 outlines the high-level sequence of steps, the recipe parameters 208 ensure that each step task within these steps is performed with precision and according to specific criteria. For example, in a DNA extraction workflow, the recipe parameters may influence: temperature settings, reagent volumes, centrifuge speed, elution volume, and quality control parameters. In an aspect, the workflow definition 206 and the recipe parameters 208 may be pre-configured and stored within the MES if the system is designed to perform the same standardized process on each sample. Additionally or alternatively, if the system is capable of handling various sample types or executing different processes, the workflow definition 206 and the recipe parameters 208 may be received when new samples are introduced, allowing the MES to tailor the workflow process according to specific test orders and sample characteristics.

In an aspect, the MES may retrieve data 210 published to LIMS, e.g., by the work cells, to inform and guide the execution of workflows. The MES may rely on this data to make informed decisions, optimize processes, and ensure that the workflow process is effectively executed. For instance, the MES may access various types of data, including: sample history data (e.g., historical data on the sample, including previous processing steps and any prior results), reagent availability (e.g., information on the availability and status of reagents, potentially including one or more of batch numbers, expiration data, storage conditions, usage rate, current reagent volumes, reagent volumes used, etc.), equipment status (e.g., data on the availability, calibration status, and, processing efficiency of the work cell, maintenance history or instruments and equipment), and current process status (e.g., real-time data on the current status of workflow processes, including which steps have been completed and any deviations or issues that were encountered). In an aspect, the MES may be configured to access LIMS continuously, periodically (e.g., every minute, hour, etc.), or in response to predetermined events (e.g., prior to each downstream instruction transmission, in response to receiving an alert, etc.).

In the converse, the MES, along with each of the other software layers, may be configured to publish data 212 corresponding to sample processing and workflow progress to LIMS. By ensuring that relevant data is accurately recorded and accessible, this interaction supports traceability, compliance, quality control, and future analysis. The MES may publish various types of data to LIMS, including one or more of: workflow process information for a sample or sample batch, sample data (e.g., sample ID/barcode information, sample batch characteristics including sample type and number of samples in the batch, etc.), step completion updates (e.g., information on the completion of each workflow step, including timestamps and details of the specific tasks performed), end-point data (e.g., final results of the completed workflow), and the like.

By leveraging the workflow definition 206 and recipe parameters 208 to form the workflow process constructed to address the test order 202, and by further considering modifications to the workflow process provided by user interactions 204 with the MES and various information associated with the system retrieved from LIMS 210, the MES may generate and transmit workflow process instructions 214 to one or more components and/or system layers to initiate sample processing. In this regard, the MES may identify an appropriate activity work cell to begin the sample processing and may transmit instructions to the conveyance platform to initiate the transport of the sample to the designated work cell. These instructions may include one or more of: the current location of the sample, the destination of the work cell, and the optimal path for transport (e.g., taking into account real-time data about the system's operational status, such as the position of other samples or materials resident on the conveyance platform, work cell status, and other potential obstacles). Responsive to receiving these instructions, the conveyance platform may facilitate the transport process to the designated work cell. Additional details regarding work cell selection and conveyance platform configuration are further elaborated upon herein.

In one aspect, the MES may directly communicate with the scheduler associated with the designated work cell. For instance, the MES may transmit, to the relevant scheduler, a variety of information including one or more of: i) an indication that a new set of samples is being sent to that work cell for processing, ii) identification information associated with those samples (e.g., sample barcode IDs), and, in some aspects, iii) a subset of the workflow process instructions (e.g., including the relevant steps and tasks that need to be performed by the work cell for the type of sample) that are associated with the stage of sample processing that the work cell is configured for. In other aspects, the scheduler may always process the samples according to the same workflow process instructions, and so the workflow process instructions may not be transmitted from the MES to the scheduler. In an aspect, the scheduler, upon detection that a sample set has arrived at the work cell, may verify that the newly arrived samples correspond to the new processing request received from the MES (e.g., by scanning a barcode (or other identifying indicia) associated with the newly arrived samples and comparing the barcode information to the sample identification received from the MES transmission). The scheduler may then facilitate sample processing in accordance with the workflow process instructions received by the MES. In other aspects, verification may just involve the scanning of a barcode or other identifying indicia without comparing the scanned information to any other data received from the MES, or elsewhere.

In another aspect, the communication between the MES and the relevant scheduler may be simpler. For instance, the MES may simply transmit an indication to the scheduler that new samples will be arriving for processing, along with the relevant sample information. The scheduler, being configured to conduct specific steps of a stage of the workflow, may automatically initiate sample processing responsive to verifying that newly arrived samples match the sample information for the new processing request provided by the MES. Alternatively, in another aspect, the scheduler may be configured to automatically initiate sample processing responsive to simply scanning the barcode or other identifying indicia associated with the received samples.

In yet another aspect, the MES may have no direct communication with the scheduler. For instance, the MES may encode sample ID information into a barcode or other identifying indicia and facilitate transport of the samples to the relevant work cell using the conveyance platform. Alternatively, in another embodiment, no encoding of sample ID information may occur by the MES. Rather, samples may be placed in trays that include pre-assigned ID information, e.g., in the form of barcodes. As the tray moves from the central storage to a work cell (e.g., via the conveyance platform), a scanner may be configured to read the ID information, automatically logging the sample's entry into the workflow. Upon arrival at the work cell, the scheduler may detect that new samples have arrived, scan them, and utilize preconfigured knowledge of the steps that the work cell was configured to perform to execute the sample processing.

The MES may receive sample processing updates 216 from one or more of the downstream layers (or from LIMS after the downstream layers publish the sample processing updates 216 to LIMS). The MES may utilize these updates to facilitate decisions that optimize the processing of samples throughout the system. For instance, in an aspect, the MES may receive an indication from a relevant scheduler when a new sample batch is received at a work cell. In some aspects, this transmission may include a confirmation that all samples in a sample batch were positively identified. In other aspects, a transmission may not be sent from the relevant scheduler when a new sample batch is received. In another aspect, the MES may receive a transmission from the relevant scheduler that the processing of all steps and tasks associated with a sample are complete, or, in other words, that a sample has been output from the respective activity work cell. In another aspect, the MES may receive transmissions from a scheduler that provide an indication of instrument status (e.g., including operational state such as online, offline, in error, etc.), maintenance alerts (e.g., notifications of instruments requiring maintenance, calibration, or repairs, which may be triaged as immediate concerns or future considerations), reagent and consumable levels (e.g., the current stock levels of critical reagents and consumables (or indications of how much has been used), low stock alerts, expiration alerts, etc.), and the like. In an aspect, the sample processing updates may be received without explicit communication with any scheduler layer. For instance, the MES may be apprised of instrument health or status, inventory considerations, and sample processing updates by accessing information in LIMS that the scheduler or instruments within the work cell have published to LIMS. Based on the updates received, the MES may make various dynamic adjustments to the workflow. For instance, if an MES is apprised that an instrument is offline for maintenance, the MES may reroute samples to an alternative work cell with similar capabilities to ensure that processing continues despite required maintenance or other disruptions. As another example, the MES may initiate automatic replenishment protocols responsive to receiving indications that reagent and/or consumable levels are low.

The second software layer, also known as the “scheduler,” functions as the step executor within the hierarchical software architecture, shepherding the samples through the various steps associated with a sample stage within an individual work cell. In general, the scheduler's primary role is to map the necessary steps to the available resources within each work cell, ensuring that the workflow process associated with the work cell is completed efficiently. In an aspect, each scheduler, or scheduler thread, may be mapped to a robotic arm that is resident within each work cell, to the work cell itself, or to a work cell type. This relationship may facilitate precise and efficient execution of tasks by leveraging the scheduler's capacity to manage workflows and the robotic arm's ability to perform physical operations. More particularly, each scheduler may be responsible for managing and arranging for the execution of steps within a specific work cell, which may involve identifying the instruments that are configured to execute the tasks in furtherance of each step. The robotic arm may be utilized to execute specific tasks and/or to ferry the samples between the instruments in furtherance of those steps. For instance, the scheduler may provide the robotic arm with instructions, such as the specific movements required to transport samples within the work cell, the timing of these movements, and the exact locations for placing or retrieving samples. This coordination may enable the robotic arm to perform complex operations with high precision. For example, to complete a series of steps in a DNA extraction stage, the scheduler may instruct the robotic arm to pick up a sample plate from an incubator, transfer it to a centrifuge, and, once centrifugation is complete, then move it to a liquid handler for reagent addition. In an aspect, the scheduler may be configured to substantially continuously or periodically monitor the progress of steps and the status of the robotic arm and other instruments within each work cell. If any issues arise, such as a mechanical fault or an unexpected delay in a task, the scheduler may dynamically adapt by identifying other available instruments that may be configured to complete the necessary tasks in furtherance of each step.

Additionally or alternatively to the foregoing, a single scheduler may be responsible for managing several work cells that perform the same overall function or similar tasks. This configuration may be beneficial for scaling operations, as it allows the system to handle larger volumes of samples while maintaining consistency across multiple work cells. In an aspect, the scheduler may allocate steps and tasks based on the current load, availability, and efficiency of the work cells it oversees. By overseeing multiple work cells, the scheduler may coordinate resource sharing, such as redistributing reagents or samples to underutilized work cells.

In an aspect, the scheduler may be configured to manage multiple batches simultaneously within a single work cell. More particularly, the scheduler may be configured to process multiple sample batches at various stages of the workflow, ensuring that each batch receives the appropriate level of attention and resources. This concurrent processing not only enhances efficiency by reducing the downtime for any given sample and/or instrument, but also allows the system to handle high volumes of samples without compromising on the precision and accuracy of individual tasks. Similar to the MES layer, the scheduler may be configured to operate independently of the overarching context of the workflow, focusing solely on the execution of specific steps within the work cell. This deliberate isolation allows it to optimize its operations without being burdened by the complexities of the entire system.

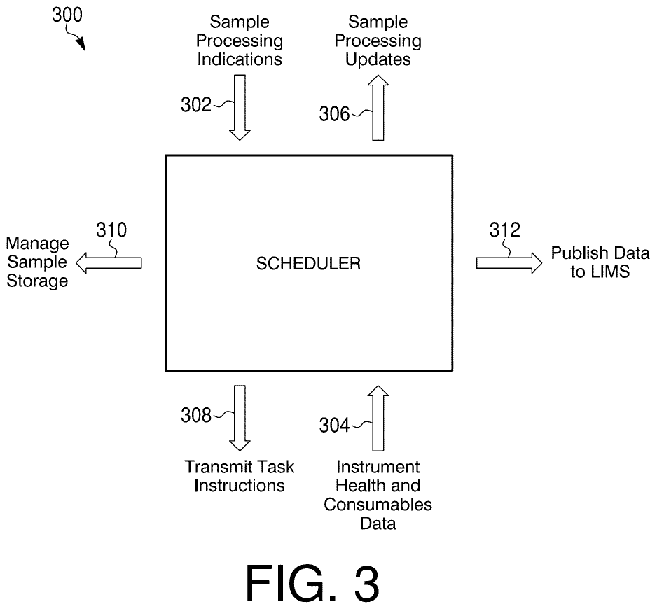

To effectively manage the sample through the execution of steps in a particular stage of the workflow, the scheduler may reconcile a variety of different types of data. For instance, referring now to FIG. 3, diagram 300 illustrates the various types of inputs and outputs that are received and transmitted by the scheduler.

In an aspect, the scheduler may receive sample processing indications 302, which act as the initial trigger for the scheduler to commence the processing of new samples. As previously discussed above in association with FIG. 2, in one aspect, these indications may be received from the MES in the form of detailed instructions associated with a particular portion of the workflow process. For instance, the instructions may include key information such as sample identification details and specific workflow process instructions tailored to the type of analysis or assay required. In another aspect, the sample processing indications 302 may be inherently deduced. For instance, a scheduler that identifies that samples have arrived at a work cell may contain preconfigured logic to automatically begin processing of those samples according to a stored workflow (e.g., which may delineate steps associated with a stage of an assay). Stated differently, the act of detecting that a sample has arrived at the work cell corresponds to the sample processing indication.

In an aspect, the collection and monitoring of instrument health and consumables data 304 promote maintenance of an efficient and reliable work cell. This data, gathered from various instruments within a work cell, may provide the scheduler with real-time insights into the operational status and readiness of the equipment. Instrument health or status data may include information on the current functioning state of each instrument, such as whether it is online, offline, or in an error state or reduced efficiency state, as well as details on any maintenance requirements, calibration needs, or recent performance issues. This information may allow the scheduler to make informed decisions about task allocation and to anticipate and address potential disruptions before they affect the workflow. Additionally, consumables data tracks the availability and levels of critical reagents, buffers, and other materials necessary for sample processing. Alerts for low stock levels, expired reagents, or other consumable-related issues, and/or requests to replenish the same, may ensure that the work cell may operate without interruption.

In an aspect, the scheduler may communicate with the MES to provide sample processing updates 306. These updates may provide real-time information about the status and progress of sample processing within the work cell. In an aspect, the scheduler may be configured to transmit these updates at various stages of the workflow, and these updates may include various types of information, including one or more of confirmations of sample receipt, detailed progress reports on the completion of each processing step, and the operational status of instruments involved in the workflow. Additionally, in some aspects, the updates may highlight any maintenance alerts, such as instruments requiring calibration or reagents running low. Through these updates, the MES may be able to make informed decisions about subsequent workflow steps, resource allocation, and any necessary adjustments to ensure smooth and efficient progression of sample processing. In other aspects, the scheduler may only communicate to the MES once a batch of samples is output from the work cell as completed. Other information, such as that described above, may be published to the LIMS, and the data may be retrieved from LIMS by the MES.

In an aspect, once the scheduler receives the sample processing indications and determines the necessary steps for each sample, it may execute these steps by allocating task-specific instructions 308 to the relevant instruments involved in each step. For instance, these instructions may include specific parameters and protocols for each task, such as one or more of volumes for pipetting, duration and temperatures for incubation, speeds and durations for centrifugation, and any other operational settings required for the step. In some aspects, the instructions may be tailored to the capabilities and current status of each instrument, ensuring that the tasks are executed within the present performance range of the instruments. It is important to note that the scheduler itself may not oversee control of the execution of any particular task. Rather, after providing the sample to the relevant instrument, the scheduler waits until the instrument has completed the operations needed to fulfill the task, before resuming control to transfer, if necessary, the sample, or cause the sample to be transferred (e.g., via the robotic arm) to the next instrument. In an aspect, within a work cell, the scheduler may be responsible for orchestrating the movement of samples to various instruments in a specific order, so that each instrument performs its designated task in furtherance of a specific step. By coordinating the sequential handoff of samples between instruments, the scheduler facilitates the smooth progression of tasks, allowing the work cell to efficiently complete a complex multi-step process.

In an aspect, the scheduler may manage the storage of samples 310 within a work cell. This process ensures that samples are maintained in the appropriate conditions when they are not actively being processed, thus preserving their integrity and quality for subsequent steps. In this regard, the scheduler may be responsible for coordinating the placement of samples into appropriate storage units, such as multi-temperature storage compartments, which may include ultra-low temperature freezers, regular freezers, refrigerators, or ambient storage units, depending on the specific requirements of the samples. In an aspect, the scheduler may manage sample storage by issuing instructions to the robotic arm to transport the samples to and from the storage units. This may involve determining the correct storage conditions based on the sample type and the stage of the workflow. For instance, certain biological samples, like DNA or RNA, may require storage at −20° C. to prevent degradation, while others may only need refrigeration at 4° C. In an aspect, the scheduler may be configured to continuously or periodically monitor the status of the storage units, ensuring that they maintain the correct temperatures, within an acceptable margin of error, and that there is sufficient capacity for incoming samples. The scheduler may also be configured to track the location of each sample within the storage units to enable the efficient retrieval of samples when they are needed for the next stage of processing, thereby avoiding delays and facilitating a smooth workflow.

In an aspect, the scheduler may record and publish data 312 to LIMS related to, e.g., sample processing, instrument performance, and workflow progression. The scheduler continuously or periodically collects and updates information as it manages the execution of tasks and steps within each work cell. This data may include e.g., one or more of: details of each operation performed (e.g., such as the time and date of task initiation and completion), the specific instruments used, the conditions and parameters under which each step was executed, any deviations or errors that were encountered, instrument health data, consumable usage or levels, reagent usage or levels, sample storage data (e.g., which samples are in storage, the designated storage compartment for each sample, the sample's location in the storage module, how long they have been in a storage module, etc.), and the like. By publishing this data to LIMS, the scheduler may ensure that all actions are accurately documented and are traceable. This comprehensive tracking may also support compliance with regulatory standards and facilitate better quality control and auditing processes. Additionally, keeping LIMS updated may enable the MES to access real-time updates on sample status, make informed decisions about workflow adjustments, and ensure that conditions are met for subsequent processing steps. Additionally or alternatively, in an aspect, the data stored in LIMS may be used for retrospective analysis, helping to identify trends, optimize protocols, and improve overall laboratory efficiency.

The third software layer 30, also known as the “instrument control layer,” exists at the instrument level and is responsible for the direct control and management of the specific tasks and operations performed by each instrument within the work cell. Unlike the higher-level software layers that handle workflow orchestration, the instrument control layer focuses on executing the specific tasks designated by the scheduler, or the tasks that it is dedicated to perform. These tasks may be broken down into detailed operations that the instrument must perform. For example, in a DNA extraction process, tasks may include pipetting specific volumes of reagents, mixing samples, incubating them at certain temperatures, or centrifuging at defined speeds. To execute each of these tasks, an instrument must perform one or more atomic operations that are executable by the instrument's hardware components.

In an aspect, the instrument control layer operates with a focused scope and does not need to understand the overall process or the sequence of tasks; instead, it concentrates solely on the specific operations that the instrument the instrument control layer oversees is tasked with performing. In an aspect, this narrow focus allows the instrument control layer to optimize the performance of individual instruments, ensuring that each operation is executed with accuracy. For instance, a robotic arm controlled by the instrument control layer may follow coordinates and timing to transport samples, avoiding the risk of human errors. Similarly, as another example, a pipetting instrument may dispense predetermined volumes of reagents from predefined supplies, adhering to predefined parameters to ensure reproducibility and consistency across all samples. The instrument control layer may also be configured to provide real-time updates to the LIMS. More particularly, as each operation is initiated, progresses, and completes, the instrument control layer records detailed data on the status and outcomes of these operations to LIMS. This recorded information may allow for tracking the progress of individual samples or batches of samples, promoting traceability, and facilitating quality control.

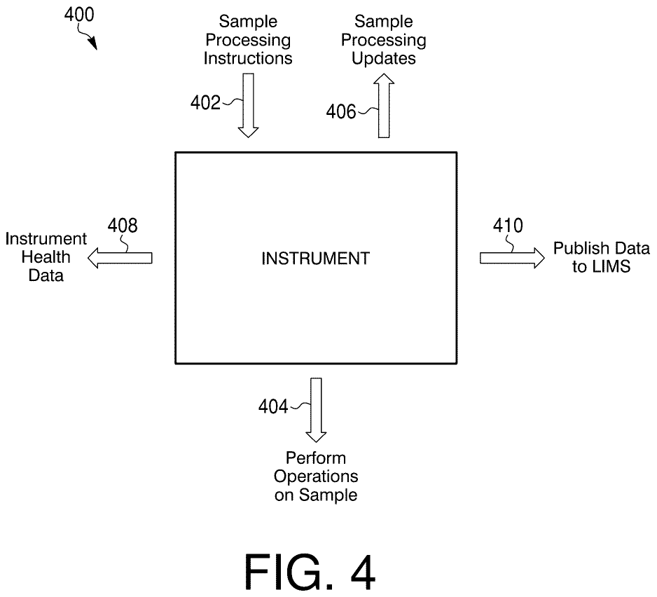

To effectively complete tasks in furtherance of the steps involved in a processing stage, the third software layer may reconcile a variety of different types of data. For instance, referring now to FIG. 4, diagram 400 illustrates the various types of inputs and outputs that may be received and transmitted by the instrument control layer.

In an aspect, the instrument control layer may receive sample processing instructions 402 from the scheduler. The instructions may be derived from the scheduler in the form of task indications that the instrument must complete. In an aspect, an instrument may be responsible for completing an entire task or series of tasks. In another aspect, the instrument may only be responsible for completing a portion of a task, the remainder of which may be completed by another instrument. In other aspects, the sample processing instructions 402 may simply be that samples are ready for the instrument to process, and the individual instrument may be designed to process the same task or series of tasks for each sample batch it receives.

In an aspect, the instrument control layer may translate the high-level workflow requirements into actionable commands for the hardware components of the instrument, which may manifest as operations performed on the sample 404. Each instrument in the work cell may be designed to carry out operations, such as, e.g., pipetting, mixing, heating, cooling, and/or centrifuging, depending on the needs of the particular step and stage of the workflow. These operations may be typically executed through a series of automated actions controlled by the instrument control layer, which interprets the instructions and converts them into specific mechanical actions. These operations may be controlled and monitored to promote accuracy, precision, and consistency. In an aspect, sensors within the instrument may provide real-time feedback to adjust parameters dynamically, promoting optimal performance and avoiding errors.

In an aspect, the instrument may transmit sample processing updates 406 to the scheduler during the execution of tasks on a sample. These updates may serve multiple purposes, including informing the scheduler of the progress and status of sample processing and facilitating real-time tracking and transparency. In an aspect, as the instrument performs its designated operations, it continually or periodically generates data, e.g., about each operation and task completion, any deviations from expected performance, and the current state of the sample. This data may be encapsulated in processing updates, which may include details such as timestamps of task initiation and completion, quantitative measurements (e.g., volumes of reagents used, temperatures maintained), any anomalies or errors encountered. In an aspect, these updates may be transmitted at the conclusion of each task, at the conclusion of a series of tasks associated with a step, at the conclusion of all tasks for a particular sample batch, or at predetermined intervals. By transmitting these updates to the scheduler, the instrument may ensure that the system remains synchronized and that any necessary adjustments may be made promptly to optimize workflow efficiency. Additionally, the scheduler may rely on these updates to make informed decisions about the execution of subsequent steps in the process. In other aspects, these details may be published to LIMS instead of or in addition to the scheduler, and if only published to LIMS, the output to the scheduler may simply be when the tasks for a sample batch are completed, or if there is an error or maintenance issue.

In addition to sample processing updates 406, the instrument control layer may also transmit instrument health data 408 to the scheduler or LIMS. The instrument health data may include, e.g., one or more of detailed diagnostics, such as error codes, sensor readings, and calibration status. If the instrument detects any anomalies or malfunctions, it may send alerts specifying the nature and severity of the issue. For instance, if a robotic arm is experiencing increased resistance in its movement or a pipette is delivering inconsistent volumes, these deviations may be logged and reported. The proactive communication may enable the scheduler to reallocate tasks to other functioning instruments or work cells, reducing downtime and maintaining the workflow's continuity.

While performing the sample processing tasks and operations, the instrument control layer may publish data to LIMS 410. This data may include records of tasks performed by the instrument, such as timestamps for the start and completion of tasks, the specific operations executed, and the results obtained from these operations. By systematically documenting each step of the sample processing workflow, LIMS serves as a centralized repository for data, ensuring that actions performed on a sample are tracked and recorded. In addition to operational data, LIMS may also receive updates on one or more of instrument health, maintenance requirements, and consumable levels. The MES may access this information in LIMS, which it may utilize to help organize the workflow process.

The fully automated system described herein is designed with a high degree of flexibility and adaptability. In some aspects, different work cells may be able to perform specific steps of a process differently based on the type of sample being processed. This capability ensures that each sample type receives the most appropriate and effective treatment, tailored to its unique properties and composition, while still achieving consistent and standardized outputs. For instance, biological samples such as blood, urine, saliva, and tissue each have distinct properties that may necessitate specialized processing methods. For example, blood samples may require lysis of red and white blood cells, followed by separation of plasma and extraction of nucleic acids from the cellular components. In contrast, urine samples, which are typically less dense compared to blood samples, have a different composition of solutes and cells, and may need different concentration and purification steps to isolate the desired analytes. To accommodate these differences, work cells may be equipped with specialized instruments, reagents, and protocols that are designed to handle the specific challenges posed by each sample type. In other aspects, the system may include multiple different work cells for achieving the same step (e.g., DNA extraction or cytosine conversion for methylation analysis via bisulfite conversion or enzymatic conversion), and some of the work cells may be appropriate for a certain sample type (e.g., blood samples), while other work cells are appropriate for a different sample type (e.g., urine) or workflows (e.g., one ending with a cancer detection report vs. one targeting minimal residual disease analysis). The MES may assign a sample of a given type to a work cell that is capable of processing that sample type. Accordingly, the MES may assign each sample to a work cell that is appropriately equipped to handle its specific processing needs, ensuring that the steps taken within each cell are optimized for the sample type.

At the same time, there may be steps that are unified across sample types for a given workflow. As an example, a workflow may be configured for cell free DNA (cfDNA) sequencing and analysis. cfDNA can be extracted from a variety of sample types including blood and urine. Blood and urine samples may begin their workflow differently, however the output of a DNA extraction work cell may be the same for both blood and urine originating samples. From the DNA extract pathway onwards, the MES may be configured to treat the blood and urine originating samples the same using the same work cells. As another example, cfDNA analysis may include methylation or targeted methylation analysis or whole genome sequencing and analysis. Therefore, after a DNA extraction work cell, samples may be selectively directed to partitioning-based partitioning work cells, bisulfite conversion work cells, work cells to introduce probes to target specific regions of the genome, or to pre-amplification work cells to prepare for whole genome sequencing. As can be seen, the flexibility and modularity afforded by the MES enables for different types of samples and different assay outputs to be processed together around a similar core of work cells in a fully automated system.

In an aspect, despite the variations in processing methods across different work cells, the system may be designed to ensure that the final outputs are consistent and meet the required quality standards. For example, the goal of DNA extraction may be to obtain purified DNA that is suitable for downstream applications, such as sequencing polymerase chain reaction (PCR) or sequencing and analysis of a variety of types (e.g., whole genome sequencing, methylation-informed sequencing, fragmentation analysis). Whether the DNA is extracted from blood or urine, as in the example above, the system ensures that the purification process yields DNA of sufficient quantity and quality. This may be achieved by tailoring the intermediate steps to the sample type while maintaining control over the final output criteria. As a result, the system may produce standardized outputs from varied inputs, making it versatile and efficient. Although the ability to handle different sample types is described herein, it is acknowledged that in other aspects, the system may only receive one sample type, and thus may not be configured to receive multiple different sample types.

In an aspect, system 100 may have the capacity to undergo improvements and adjustments to instruments and hardware components while remaining “online,” or operational. This capability may allow for continuous system functionality and minimal disruption to ongoing processes to maintain high throughput and efficiency in laboratory environments. In an aspect, when an instrument or hardware component requires maintenance, upgrades, or adjustments, the MES may dynamically reallocate tasks to other available work cells or instruments. For example, if a specific automated pipettor in a work cell needs calibration, the MES may temporarily route samples to an alternative work cell that is configured for a similar process flow, thereby ensuring that the overall workflow remains uninterrupted. Similarly, if a work cell is in need of maintenance or repair and needs to be taken offline for a period of time, then samples may be directed by the MES to alternative work cells of the same type so that sample processing may continue, albeit with potentially reduced capacity, while avoiding work stoppage of the entire system. This dynamic task reassignment may allow for real-time adjustments without halting the entire system.

Furthermore, the modular design of the system supports the addition or removal of hardware components without necessitating a complete shutdown. Specifically, new instruments may be integrated into existing work cells, and software updates may be deployed to enhance performance or introduce new functionalities. In an aspect, the scheduler layer, which manages steps within individual work cells, may incorporate these changes, updating its task mapping to include the new or modified components. For instance, given a scenario where a new type of reagent dispenser is introduced in a work cell to improve sample preparation, the MES or the scheduler may be apprised of the updated functionality associated with the work cell and may thereafter update the workflow processes to utilize the new dispenser. As another example, new work cells may be integrated into the system, e.g., to increase the system's capacity, or software updates may be pushed out to one or more work cells. The MES, which manages the allocation of samples to individual work cells, may incorporate these changes, updating its task mapping to include the new or modified work cell. For instance, given a scenario where a new work cell is introduced, the MES may be apprised of the new work cell or updated functionality associated with the work cell and may thereafter update the workflow processes to utilize the new or updated work cell. As such, multiple versions of the same kind of work cell (e.g., Extraction v1 and Extraction v2) can operate together, with the MES being able to treat them as identical units so long as the input and output requirements are the same, or at least compatible.

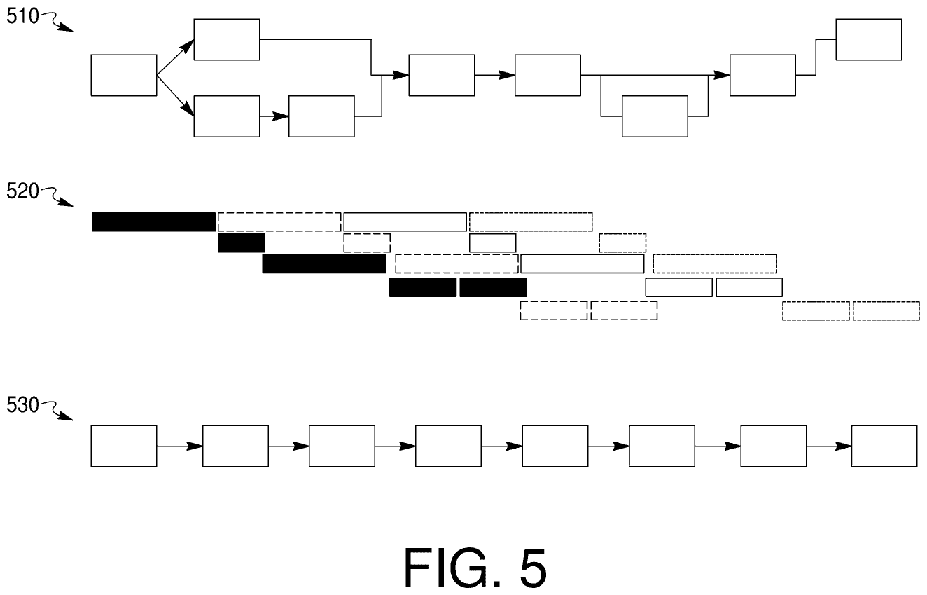

Referring now to FIG. 5, the provided diagrams depict processing structures illustrative of the typical decisions that may be made and variations in processing introduced by the various layers of the hierarchical software architecture 100. Diagram 510 is illustrative of the processing and decision structures that may be made by the first software layer 10. The diagram 510 shows that the workflow generally follows a core path or trunk with possible deviations that may be based on the type of sample, the type of assay, the expected output, and a variety of other factors as described herein. Each box in the diagram 510 may be representative of a work cell or a step in the workflow to be performed with or on a sample. Diagram 520 is illustrative of the processing and decision structures that may be made by the second software layer 20. The diagram 520 is similar to a Gantt chart and relays the interdependencies between various components on a given work cell (represented by the horizontal axis) that a scheduler must consider when allocating work for the work cell components. Additionally, the diagram 520 provides a temporal aspect (represented by the vertical axis) that illustrates how each task takes a specific amount of time that must be considered when determining how to most efficiently perform tasks on samples given the resource constraints. Diagram 530 is illustrative of the processing and decision structures that may be made by the third software layer 30. Because the third software layer 30 relates to the control of individual instruments in a work cell, the boxes in the diagram 530 can be considered the operations that each instrument performs during the work flow. Diagram 530 is the simplest of the three diagrams because while the operations performed by the instrument may be complex, the order of operations is straightforward and dictated by the scheduler.

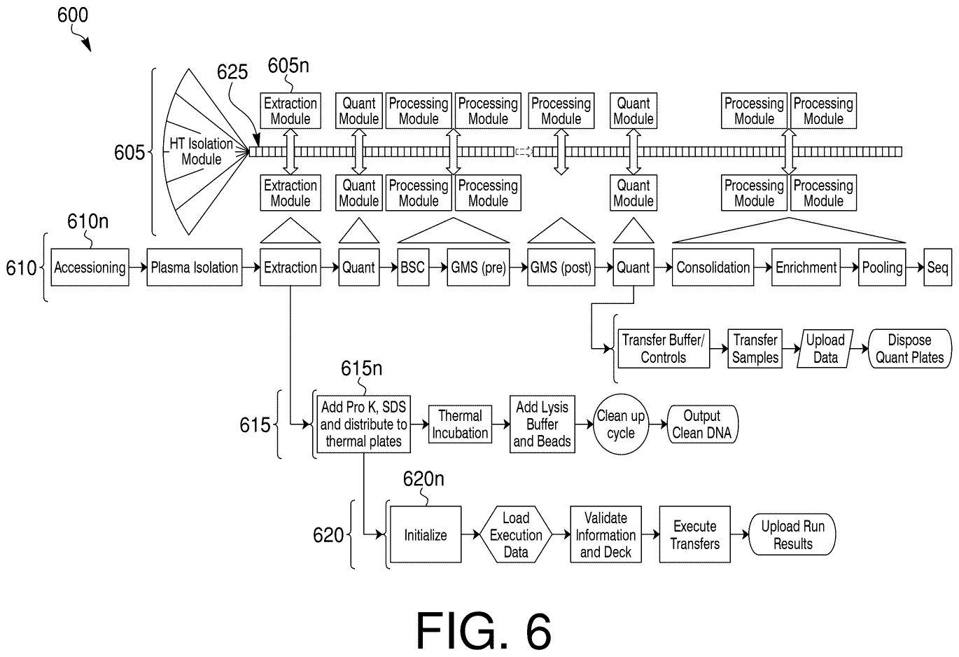

Referring now to FIG. 6, the provided diagram 600 depicts an exemplary structure and workflow of an automated laboratory system. Section 605 of diagram 600 illustrates an exemplary logical layout that shows a series of different types of work cells 605n that are arranged along a central conveyance platform 625 that facilities the movement of samples between different stages of the workflow. More particularly, the MES may control the transport of samples through the available work cells based on the workflow process. Below the logical layout section 605, section 610 outlines the stages of sample processing performed by the work cells shown in the logical layout section 605. Specifically, each stage 610n represents a phase in the processing of samples, such as isolating plasma, extracting nucleic acids, quantifying samples, and preparing them for sequencing mapped, in this example, to a particular work cell type. The scheduler layer section 615 provides a detailed breakdown of the steps 615n managed by the scheduler within each stage 610n. More particularly, once the samples are transported by the MES to a particular work cell, the scheduler, which may be specifically associated with that work cell, may be responsible for executing the steps involved in the stage of the workflow that the work cell is configured to execute. For example, as illustrated, during the extraction stage, the scheduler may oversee tasks like adding Proteinase K and SDS, distributing samples to thermal plates, thermal incubation, adding lysis buffer and beads, and running a clean-up cycle to output clean DNA. Section 620 may be representative of the instrument control layer, which details the specific tasks 620n performed by the individual instruments within each work cell. More particularly, the scheduler layer may employ instruments to accomplish tasks in furtherance of each step. For instance, during the “add Proteinase K and SDS” step identified in section 615n, one or more instruments may perform the various tasks involved in the step completion, including: instrument initialization, the loading of execution data, the validation of information, the execution of transfers, the uploading of run results, and the disposing of quant plates. The structure and workflow of an automated laboratory system depicted in FIG. 6 is merely given as an example. Consistent with the principles and techniques described herein, a variety of stages, processes, tasks, and operations are anticipated to be performed in the handling of any given sample to achieve the aims of any given assay.

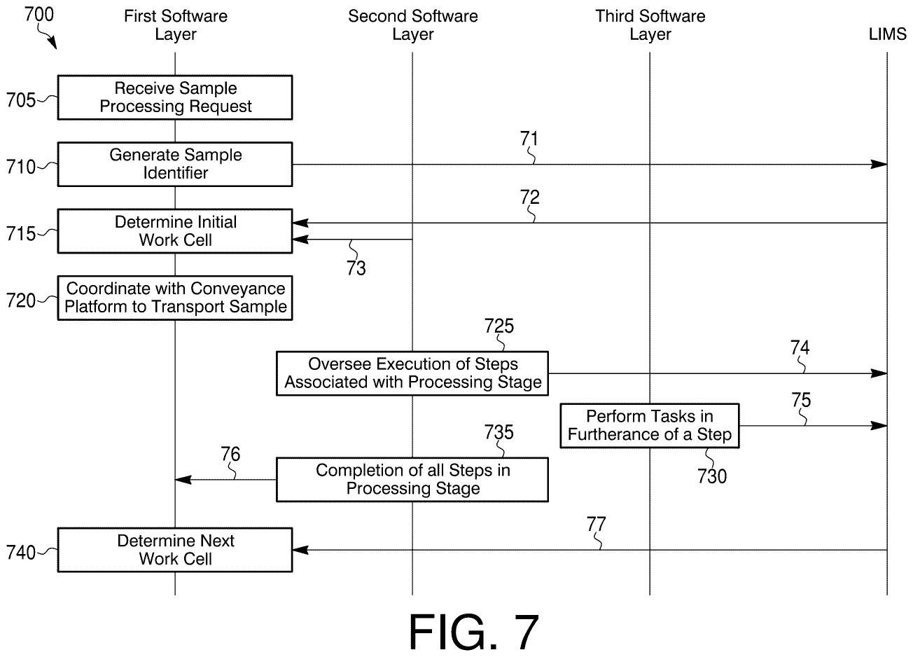

Referring now to FIG. 7, diagram 700 presents exemplary general steps involved in processing a new sample request and identifies the software layers that are responsible for handling each task.

At 705, the MES layer may receive a request to process a new sample or a new batch of samples, which may need to be processed according to a particular workflow process. The request may be received or initiated during accessioning of the sample or batch of samples. In an aspect, subsequent to receipt, the MES layer may log pertinent details about the sample, including its type, the specific assays or analyses required, and/or any special instructions or desired outputs specified by the user or workflow process. In other embodiments, the MES layer may simply receive the request to process the new sample and may not need accompanying information, because each sample or batch of samples may be processed in the same way.

At 710, subsequent to receipt of the sample processing request, the MES layer may generate a unique identifier for the sample, e.g., in the form of a barcode, a Radio-Frequency Identification (RFID) tag, QR code, or the like. This unique identifier may serve as a digital fingerprint for the sample, promoting accurate tracking and management throughout the processing workflow. In an aspect, the MES layer may generate this identifier utilizing details contained in, e.g., one or more of: the initial sample processing request (e.g., such as the sample type, specific assays required, and any special instructions), the workflow process (e.g., stored in local storage), information obtained from LIMS, and the like. The identifier may be physically attached to or inscribed on the sample container or a carrier, ensuring that every subsequent interaction with the sample is accurately recorded and attributed. Alternatively, a sample received by the system may already have a unique identifier assigned to it, e.g., in the form of a barcode, RFID tag, QR code, or the like, and the identifier may simply be scanned upon entry into the system and at different locations within the system. In this aspect, the MES layer may not generate a unique identifier for the sample, and step 710 may instead simply include scanning a sample identifier or otherwise associating the sample identifier with the sample. In an aspect, the sample carrier is already associated with a unique identifier and the MES layer associates the unique identifier for the sample container carrier with the unique identifier for the sample for the purpose of tracking the sample during processing.

In an aspect, this unique identifier may be used for maintaining the integrity and traceability of the sample within the system. In an aspect, upon creation, details of the unique identifier may be published to LIMS, e.g., as illustrated by arrow 71 in FIG. 7. As the samples progress through the processing stages, the unique identifier may, in some aspects, facilitate communication and data exchange between various software layers and physical components involved in the workflow, or may provide a record in LIMS of a sample's movement through the workflow for later review. For instance, as the sample progresses through different work cells and undergoes various processing steps, each action may be logged against the identifier in LIMS. This comprehensive logging may facilitate tracking of the sample's journey, from initial preparation to final analysis, enabling real-time monitoring or retrospective analysis, if needed. Moreover, the unique identifier may play a role in coordinating the autonomous actions of the system's robotic components and instruments. Specifically, as the sample moves through the workflow, the identifier may be scanned at various steps, tasks, and operations, which, in some aspects, may trigger the relevant software layer to execute the appropriate actions. In other aspects, even if certain actions are not triggered and the identifier is simply recorded to LIMS upon scanning of the identifier, a record of the sample's path through the system is recorded. This coordination and record keeping may not only enhance efficiency but also reduce the potential for human error, ensuring that the right processes are applied to the right sample at the right time.

At 715, once the sample has been uniquely identified and logged into the system, the MES may analyze the details of the processing request, which may include data designating the type of sample, the required assays, and any other specific instructions provided. In other aspects, the MES may perform the same processing steps on all samples received by the system. The MES may determine the most suitable work cell to begin the sample processing process. This determination may involve, e.g., one or more of consulting the local workflow process, accessing LIMS to obtain data regarding the published activity and health of various work cells within the system, as represented by arrow 72, and/or considering updates received from the lower system layers (e.g., notifications provided to the MES or LIMS by the scheduler regarding the status of processing within the respective work cell), as represented by arrow 73. For instance, the MES may, e.g., assess the status of each work cell, considering which are currently operational, which are undergoing maintenance, which are occupied with other tasks, which have sufficient supply of consumables, and which are over or under burdened. Additionally, the MES may be apprised of the types of instruments, their conditions, and the reagents or other consumables available in each work cell, or may receive this information from LIMS.

At 720, upon determining the appropriate initial work cell to transfer the sample to, the MES may coordinate with the conveyance platform, which may consist of various components such as one or more of conveyor belts, tracks, pucks, containers, drones, robots, etc., to execute the transport. More particularly, the MES layer may send a set of detailed instructions to the conveyance platform, specifying the current location of the sample, the designated work cell, and the path for transport. In an aspect, these instructions may take into account real-time data about the system's operational status, including the position and availability of other samples, work cells, consumable supplies, and any other potential obstacles. In one aspect, as the sample is moved, its unique identifier may be scanned and tracked (e.g., by sensors integrated in or situated along the conveyance platform), or the sample may simply be scanned and tracked when it is put onto the conveyance platform and when it is removed from the conveyance platform (e.g., by a robotic arm). This real-time tracking may allow the sample's location to be known as it moves through the system, allowing the system to monitor or record the transport process closely and, in some aspects, to intervene if any issues arise. Alternatively, in another aspect, the sample may only be scanned upon arrival at the designated work cell.

At 725, upon arrival at the designated work cell, a scheduler, which may be mapped, e.g., to a robotic arm, work cell, or work cell type, may assume control and ensure that the processing steps associated with a particular stage are carried out. In an aspect, each sample processing stage may encompass a wide range of steps depending on the specific requirements of the workflow and the capabilities of the work cell. These steps may include preparation steps such as lysis, centrifugation, conversion, and elution, analytical steps such as PCR or sequencing, or finishing steps such as purification or quantification. The scheduler layer may identify the necessary steps for the sample at this stage, allocating the sample to the appropriate instruments within the work cell at appropriate times. For example, if the work cell is configured for DNA extraction, the scheduler layer may direct the sample through the series of extraction steps, utilizing equipment like lysis stations, centrifuges, and pipetting robots as needed, which may collectively perform the necessary tasks in furtherance of each step 730. In an aspect, to reduce the risk of contamination or error, the scheduler layer may leverage feedback from one or more of, e.g., sensors and instruments. For instance, barcodes on sample containers may be scanned at each step to verify the sample's identity and ensure it follows the correct processing path (not illustrated in FIG. 7) and that the correct reagents are introduced to the sample. In other aspects, as described above, the identity may not be verified but may be recorded at the various steps.

Upon completion of each step and each task, one or both of the scheduler layer and instrument control layer may publish event data to LIMS, as indicated by arrows 74 and 75, in order to document the workflow process to promote traceability and allow for quality control checks.

At 735, upon completion of all processing steps within the work cell, the scheduler may output to the MES, and, in some aspects, to LIMS, that the process to be performed by that work cell is completed. Upon completion at a given work cell, the MES may determine the next processing step. More particularly, the scheduler may transmit an indication, represented by arrow 76, indicating that all steps in the current processing stage are complete. Subsequently, the MES may consult the predefined workflow process to identify the next processing step. Once the next processing step is identified, the MES may determine, at 740, the most appropriate work cell to execute this step. This may involve consulting LIMS, represented by arrow 77, checking the availability and load of potential work cells, ensuring that the selected work cell has the necessary instruments, reagents, and capacity to handle the sample. The MES may additionally consider factors such as maintenance schedules, current queue, and the specific capabilities of each work cell to optimize resource allocation and efficiency. Additionally or alternatively, the MES layer may consider the current queue and workload distribution across the system, aiming to balance the workload among various work cells to avoid any single work cell from becoming a point of congestion. This may involve dynamically adjusting assignments based on, e.g., real-time data or predictive analysis. In some aspects, the work cell determined to be most appropriate may be published to LIMS. In some situations, the MES layer may determine that the best work cell to process the next step is the current work cell, in which case processing of the next step may begin at the existing work cell, if that work cell is able to process samples for more than one step. In other situations, the MES layer may determine that another work cell, different than the current work cell, is the best work cell. For example, each work cell may only be able to perform processes for one given step in the overall workflow. In this situation, the MES may initiate the transport process by interfacing with the system's conveyance platform. The identifier for the sample may be scanned upon one or more of being output from the work cell, being loaded onto the conveyance platform, or being received by the subsequent work cell. In an aspect, the prior steps may repeat until the processing workflow is complete.

Following the completion of processing (e.g., through all of the work cells), the sample may undergo one, or a series, of final handling procedures. If further analysis or storage is required, the system may coordinate the transfer of the sample to an appropriate storage unit, where it may be maintained under optimal conditions until needed. Alternatively, if the sample is no longer required, it may be disposed of according to established safety and environmental protocols. In some aspects, the MES, LIMS, or another component, may generate and distribute reports containing the results of the analysis to the relevant stakeholders, such as laboratory technicians, researchers, or external clients. These reports may be customized to include specific data outputs, quality metrics, and/or any additional insights gleaned from the process. In some aspects, the processed samples may be output from the system in a container suitable for further downstream processing.

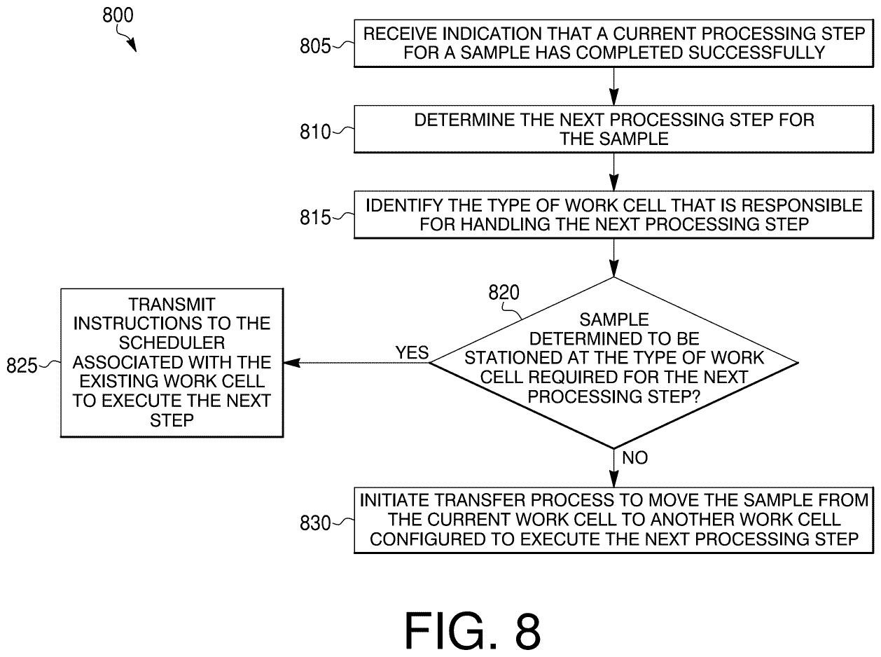

Referring now to FIG. 8, an exemplary flow 800 is provided that delineates a decision-making process for work cell assignment. Exemplary flow 800 may be facilitated using some or all of the components and processes outlined in the fully automated system that employs the hierarchical software architecture 100.

At step 805, the MES may receive an indication that a sample's current processing step at a given work cell has been completed successfully. In an aspect, this indication may be received directly from the scheduler for that work cell, e.g., via transmission of a completion signal from the scheduler to the MES. In an aspect, this completion signal may or may not include various information in addition to completion, e.g., including sample ID, work cell ID, timestamp of completion, and/or the status of the process. Additionally or alternatively, in another aspect, the MES, upon receipt of the completion signal, may access LIMS to verify the sample's status and confirm that all required operations that are needed for step completion have been performed, although this step is not necessary. This cross-checking of the completion signal, if performed, may promote accuracy and consistency, confirming that the sample ID and the completed stage match the recorded data. In some aspects, the MES may update LIMS to reflect the completion of the current stage. Additionally or alternatively, in another aspect, the MES may not receive any completion signal from the scheduler and may dynamically determine that a sample stage is complete. For example, the MES may be configured to access the data stored in LIMS continuously or at predetermined intervals and confirm that the published data in LIMS indicates completion of the relevant sample stage.

At step 810, the MES may determine the next processing step for a given sample, ensuring that the sample proceeds correctly through the necessary stages of analysis and/or preparation. In one aspect, the MES may retrieve, e.g., from LIMS, the current status of the sample. This status may include details such as one or more of the sample ID, the last completed step, the work cell from which the sample is being output as completed, and any other pertinent data associated with the sample's progress. Next, in an aspect, the MES may access the workflow process associated with the type of sample being processed, if multiple sample types are able to be processed by the system. This workflow process may outline the sequence of steps the sample must undergo based on its type and the desired end goal, such as DNA sequencing, PCR, or clinical diagnostics. In other aspects, the system may perform the same workflow process for all received samples, and thus knowing which work cell the sample has completed processing at may be sufficient information to determine the next stage to be performed. Using the current status information, in one aspect, the MES may identify the next stage in the workflow for the sample by consulting the workflow process to determine the subsequent step following the just-completed step. In an aspect, the MES may also evaluate any conditional steps in the workflow that depend on specific criteria or results from previous stages. For example, if the DNA yield from extraction is above a certain threshold, the sample may proceed to quantification, whereas if the DNA yield from extraction is below the threshold, a re-extraction process or other corrective steps may be necessary. Once the appropriate next stage is identified, the MES may update LIMS to reflect the new step that the sample is moving to.

At step 815, the MES may identify the type of work cell that is responsible for handling the next processing step. While FIG. 8 depicts steps 810 and 815 as two separate steps, these steps may be one single step, or step 810 may not need to be performed, e.g., if the system processes all samples in the same way. In one aspect, the MES may consult the workflow process, which may specify the specific type of work cell that should process each step. In another aspect, the MES may identify the type of work cell responsible for handling the next processing step dynamically. For instance, the MES may access a database that includes information on the tasks each work cell may be capable of performing, the instruments and reagents available at each work cell, and/or the current load or queue of samples being processed at each work cell. The MES may match the requirements of the next processing step with the capabilities of the available work cells to identify the type of work cell that is configured to effectively complete the next processing step.

At step 820, the MES may determine whether the sample is presently stationed at the type of work cell required for the next processing step. In an aspect, knowing the type of work cell needed to handle the next processing step (as identified in step 815), the MES may determine whether the sample is stationed at that type of work cell. If the MES determines, at step 820, that the sample is already at the appropriate work cell, then it may transmit, at step 825, instructions to the scheduler associated with the existing work cell to execute the next step without relocating the sample. In an aspect, the MES may verify that the work cell is fully prepared for the next step, which may include confirming that all required instruments and reagents are available and operational. Once readiness is confirmed, the MES may generate or access predetermined instructions for the scheduler, outlining the operations to be performed during the next step. In some aspects, an individual work cell may only be configured to perform operations to complete a particular step, rather than being capable of performing different steps. In this case, the MES may know that the current work cell is not the appropriate next work cell, and step 820 may be skipped.

If the MES determines, at step 820, that the sample is not currently present at an appropriate work cell, then it may initiate, at step 830, a transfer process to move the sample from its current work cell to an appropriate work cell associated with the next processing step. In an aspect, this may involve a comprehensive decision-making process to identify the most suitable work cell and/or work cell type for the next stage of processing. More particularly, this decision-making process may involve analysis of one or more factors to promote optimal efficiency and resource utilization. For example, the MES may identify all work cells capable of performing the required tasks for the next stage. This may involve consulting the workflow process to identify the specific requirements of the upcoming step and matching those with the capabilities of the available work cells. Thereafter, the MES may evaluate the availability of these identified work cells, which may include assessing one or more factors. For instance, in an aspect, the MES may check the current operational status of each identified work cell, ensuring that they are online and not engaged in other processes that may prevent their immediate, or near-term, use. Additionally or alternatively, in another aspect, the MES may also assess the resource levels of each work cell, verifying that they have sufficient reagents, consumables, and any other necessary materials to complete the next step without interruption. Additionally or alternatively, in another aspect, the MES may consider the maintenance schedules and any downtime that may affect the availability of each of these work cells. In an aspect, once the MES has gathered the relevant information, it may select the optimal work cell for the next step based on a combination of these factors. Thereafter, the MES may initiate the transfer of the sample to the chosen work cell, coordinating with the conveyance system to ensure safe and accurate movement. Ultimately, the goal of this process is to select a work cell that not only meets the technical requirements but also operates reliably and efficiently. It seeks to balance the workload across different work cells, avoiding bottlenecks and minimizing wait times for each sample. This load balancing helps to ensure that no single work cell becomes a point of delay in the workflow, thus maintaining overall system efficiency. Although terms like the “best” or the “optimal” work cell or instrument are described herein, it is understood that more than one work cell or instrument may be reasonably able to handle the next sample, and thus there may be more than one “best” or “optimal” work cell or instrument, and the MES or scheduler may select one as the next work cell or instrument to assign the sample to.

The system is designed with flexibility to accommodate various experimental and assay needs, allowing steps (e.g., those performed in association with FIGS. 7 and 8) to be performed out of order, added, or omitted as necessary. This adaptability allows the system to handle a wide range of laboratory workflows with the same level of accuracy and traceability.

Additionally, certain processes may be repeated one or more times to ensure the desired level of precision and accuracy, such as when additional analysis is required or when iterative refinement of results is necessary. This dynamic approach enhances the system's capability to manage complex and diverse laboratory tasks efficiently.

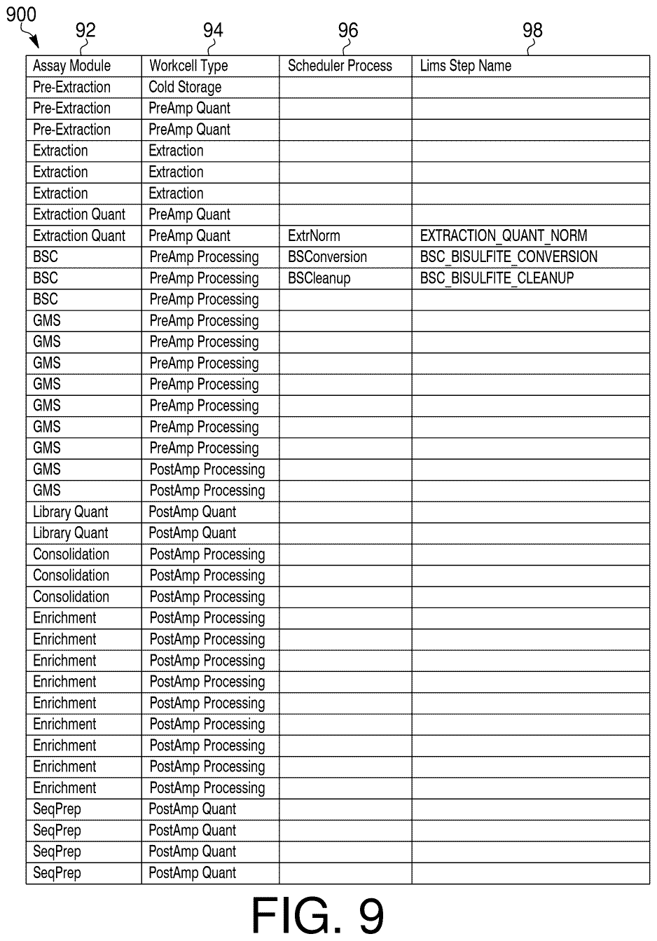

To complement flow diagram 800, FIG. 9 presents diagram 900 that provides a detailed mapping of a subset of the various stages of the workflow 92, work cell types 94, steps to be performed by a work cell in a specific stage of the workflow 96, and LIMS step names 98 involved in an exemplary workflow process. It should be noted that the specific mapping is given merely as an example, and in alignment with the principles and techniques described herein, the mapping may be modified to accommodate additional stages, steps, and tasks as required by different sample types, assaying techniques, analytical goals, etc. By using the detailed protocol information, the MES may efficiently manage sample workflows through the hierarchical software structure, promoting precise and autonomous processing of biological samples. As a non-limiting example of this process, suppose that the MES receives an indication that a sample has just completed the ExtrNorm step. The MES may identify that the next step for the sample is BSConversion. As indicated by diagram 900, this step necessitates a PreAmp Processing work cell, indicating that the sample must be relocated to a different work cell designed for pre-amplification processes. The MES then evaluates the available PreAmp Processing work cells, considering factors such as current load, availability, and predefined heuristic criteria to select the optimal work cell for the next step, as previously described above in relation to FIG. 8. After selecting the appropriate PreAmp Processing work cell, the MES may initiate the transfer of the sample. This may involve coordinating with the conveyance system to ensure the safe and accurate movement of the sample from the PreAmp Quant work cell to the PreAmp Processing work cell. The MES may then receive an indication when the sample has completed the BSConversion step. The MES may identify that the next step for the sample is BSCleanUp. Since BSCleanUp requires a PreAmp Processing work cell, the sample may remain at the current work cell to complete the step. However, the MES may identify that another PreAmp Processing work cell is more optimal for performing the BSCleanUp stage for the sample and may initiate the transfer of the sample to the other PreAmp Processing work cell as described.

In an aspect, it may be possible to perform each stage of the process (e.g., all extraction steps, all quantification steps, all pre-amplification steps, etc.) in one continuous sequence (e.g., without segmentation into the defined steps illustrated in FIGS. 8 and 9 above). However, one potential feature of system 100 is its ability to rebalance tasks after the completion of up to each step. This dynamic rebalancing capability may maintain system efficiency and allow for regular maintenance cycles without disrupting the overall workflow. For instance, in an aspect, during processing of a step, the scheduler layer may receive an indication (e.g., from a human operator, the MES layer, etc.) to halt operations after the completion of the step so that a maintenance cycle may be implemented. The scheduler layer may transmit an indication to the MES indicating that it is going to be offline for maintenance and, based on this information, the MES may redistribute upcoming tasks to different work cells as needed. Or, a given work cell may have sufficient reagents and consumables to perform one step within a given stage, but not all of the steps within a given stage. The MES may still assign samples to that work cell for the steps that the work cell still has sufficient supplies to perform, but may reallocate the sample to a different work cell to perform the steps that the work cell has insufficient supplies to perform. As another example, an instrument required for a certain step may be offline or require maintenance in a particular work cell, but the work cell may have the needed instruments to perform a different step, and thus the MES may allocate samples to the work cell to perform the step that the work cell does have the needed instruments to perform.

In an aspect, if the samples being handled by a work cell that is slated to go offline still need to go undergo subsequent steps, these samples may either be placed in temporary storage (e.g., available in the present work cell), or may be transported to another relevant type of work cell that may be able to continue the sample processing. For example, consider the extraction stage in the workflow process in FIG. 9. After completing the “BSConversion” step in FIG. 9, the MES layer may decide, based on various factors, to take one of the PreAmp Processing work cells offline for maintenance. The remaining preamp processing steps, e.g., “BSCleanUp”, etc., may be reallocated to other available PreAmp Processing work cells. This dynamic rebalancing avoids bottlenecks or interruptions in the workflow and allows the system to be able to handle high throughput efficiently.

In an aspect, system 100 may be configured to handle and address various types of errors that may occur in a work cell. More particularly, the high-volume capacity of system 100 may come with acknowledgement that some number of errors are inevitable in a high-throughput environment, and the system may be configured to prioritize maintaining uptime and workflow continuity, while creating back-up protocols to inhibit sample waste if an error occurs. Accordingly, in an aspect, system 100 may employ a tiered-approach to error resolution. For example, when an error occurs in a work cell, the relevant scheduler layer may detect the issue and pause the workflow. The scheduler's immediate response may not be to discard the sample(s), but to hold its processing, preserving the material in its current state and/or to move the material to the appropriate multi-temperature storage compartments. In an aspect, the scheduler may initially attempt an automatic retry of what was occurring when the error was reported for common, transient errors, such as communication issues or minor instrument glitches. This retry mechanism may be encoded in the software with specific timeouts and retry limits.

If the automated retry fails, system 100 may send a digital alert to the first tier of support staff. These ground-level laboratory technicians may be trained to handle frequent and straightforward errors. Their role may be to quickly assess and resolve the issue, such as by directing the operation manually under close supervision. The technicians may evaluate the error based on predefined criteria, such as the type of error, the integrity of the sample material, and/or the operational readiness of the instrument. If the issue can be resolved quickly and the material is unaffected, the technician may retry the operation. If the error is more complex or beyond their capability, they may escalate the issue to the next tier of support. Specifically, more complex errors may be handled by a higher-tier technical team, e.g., IT or an engineering team, with expertise to diagnose deeper issues within the scheduler or instruments. This team may conduct a detailed assessment to determine the root cause of the error, the feasibility of its resolution, and the broader impact on the workflow and system efficiency. If the technical team resolves the issue and the sample is deemed intact, they may resume processing. However, if the error cannot be resolved quickly or the sample is compromised, they may decide to discard the sample. This decision may be based on the severity of the error, the viability of the sample, and the need to optimize resources while maintaining high throughput. Accordingly, while the system incorporates some automatic retry mechanisms, it may, in certain situations, ultimately rely on human expertise for more challenging issues. This blend of automation and infrequent human intervention allows the system to handle errors dynamically, maintaining high throughput and efficiency without compromising on the quality of the output.

In an aspect, real-time inventory monitoring may be achieved through sensors integrated into the work cells, which may provide continuous or periodic feedback to the system, enabling inventory tracking. Inventory data may be logged in LIMS, maintaining an up-to-date record of all materials used and required by each work cell. The system may set a predefined threshold for each inventory item, and when the quantity of an item falls below its threshold, the system may automatically initiate replenishment protocols. This process may involve checking for available stock in the central inventory storage, or in a plurality of inventory storages, generating automated requests for additional supplies, and coordinating the delivery of supplies to the respective work cells without interrupting, or with little interruption to, ongoing processes. In an aspect, the system may be configured to perform restocking of materials during downtime periods, ensuring that sample transfer and processing is not delayed due to the distribution of essential supplies to the work cells. More particularly, during periods when the conveyance platform, or at least a portion of the conveyance platform, is not occupied with sample transport, such as between processing cycles or scheduled maintenance windows, the system may be configured to strategically leverage these intervals to move reagents, consumables, and other necessary materials to the appropriate work cells. For example, once it is detected that a supply of consumables within a work cell has fallen below a threshold level, the MES may be notified, or the MES may observe the low level via LIMS, and the MES may transport the consumable(s) that have fallen below the threshold level to the relevant work cell the next time the MES determines that samples do not need to be conveyed along the route to the work cell. This proactive approach may ensure that each work cell is sufficiently stocked and ready to handle its assigned tasks without interruptions, thereby promoting continuous workflow operations. Additionally, by synchronizing restocking activities with the conveyance platform's idle times, the system maximizes efficiency and inhibits bottlenecks caused by waiting for supplies.

In an aspect, the system may dynamically assign tasks to work cells based on current inventory levels and sample processing requirements, ensuring that work cells with sufficient inventory are prioritized for tasks that require those materials. The MES layer may be configured to adapt to changes in inventory status, reassigning tasks to alternative work cells if necessary to maintain continuous processing. By managing inventory replenishment in parallel with sample processing, the system may significantly reduce downtime. Work cells may remain operational without waiting for manual inventory checks and replenishments, allowing for continuous operation that promotes high throughput and efficiency.

As an example of the foregoing, in an exemplary scenario, the system may be processing a high volume of blood samples for DNA extraction and subsequent sequencing. Blood samples may arrive and may be assigned to specific work cells for initial processing. Work cells equipped for DNA extraction may begin processing the samples, with real-time monitoring ensuring that extraction reagents are available throughout the process (for example by monitoring the extraction reagents logged as used by the work cell in LIMS or by monitoring a reagent level tracked by measuring devices such as scales and provided to the schedulers or to LIMS). As reagent levels drop, the system may automatically initiate a replenishment request, and additional reagents may be delivered to the work cells without interrupting the extraction process. While DNA extraction is ongoing, other work cells may conduct other steps of the workflow, such as preparing samples for sequencing, including the preparation of sequencing libraries and loading samples onto sequencing machines. As DNA extraction completes, samples may be transferred to sequencing work cells, with the system ensuring that all necessary materials for sequencing are in place, inhibiting any delays. The system may continuously or periodically monitor and manage both sample processing and inventory, ensuring efficient and uninterrupted operation. For example, the scheduler may report this information to the MES layer, or the scheduler or instruments may report this information to LIMS, and the information may be accessed in LIMS by the MES, or both.

In an aspect, prioritization algorithms may be employed to evaluate the urgency of inventory replenishment requests. These algorithms may consider various factors, such as one or more of the current stage of the assay, the importance of the reagent or tool for subsequent steps, the number of other work cells available to perform this step, and the overall impact on the workflow if the item is not replenished promptly. In an aspect, the system may dynamically adjust the priority of inventory requests based on real-time data. For instance, if a reagent critical for a high-priority assay step, or a step that fewer work cells are able to perform, is running low (e.g., as a result of normal use, an unexpected spill, etc.), the system may elevate the priority of the replenishment request for that reagent over other less critical items.

In an aspect, upon detecting an urgent inventory need, the system may initiate the replenishment process. This may involve generating an automated request to the central inventory management system, one or more disbursed inventory systems, or external suppliers, specifying the urgency and required quantities. The system may coordinate the expedited delivery of critical reagents and tools to the specific work cell where they are needed. This may involve prioritizing the use of fast transport mechanisms, such as robotic conveyance tracks or even emergency manual intervention, to ensure timely delivery. The system may optimize resource allocation by temporarily reallocating available inventory from less critical work cells to those with urgent needs. This ensures that critical processes continue without interruption while awaiting the arrival of new supplies.

In an aspect, the hierarchical software architecture described herein may be extended to support geographically distributed laboratory or manufacturing environments, e.g., in which multiple remote facilities or workspaces operate collaboratively as part of a unified autonomous workflow management system. In such aspects, each facility or workspace may function as an independent node within a larger networked system, with each node containing its own set of some or all of: activity work cells, buffer work cells, instruments, multi-temperature storage, a corresponding second software layer (scheduler), and local implementations of the third software layer for instrument control. In an aspect, a top-level instance of the MES, or a federated set of MES components, may be configured to orchestrate a workflow process across the multiple physically separated or workspace facilities. More particularly, the MES may determine whether a given stage of the workflow process is to be executed at the local facility or workspace in which a sample resides or whether downstream processing should occur at a different facility or workspace based on inter-facility operation considerations.

In these distributed implementations, the MES may dynamically allocate stages of the workflow process among remote nodes based on one or more factors such as site-specific capacity, instrument and work cell availability, reagent and consumable inventory levels, environmental requirements for particular processing steps, real-time component health indications, geographic proximity, or planned maintenance activities. In place of, or in addition to, the conveyance platform used for intra-facility sample movement, the system may incorporate a logistics coordination subsystem that serves as the inter-facility functional equivalent of the conveyance platform. This logistics coordination subsystem may coordinate the physical transfer of samples, materials, consumables, or partially processed intermediates between facilities, while also managing the secure and synchronized transfer of workflow state information, sample identifiers, and associated metadata. In some aspects, the logistics coordination subsystem may interface directly with the MES to schedule inter-facility transfers, track samples and materials while in transit, manage chain-of-custody requirements, and ensure that relevant LIMS repositories at each facility or workspace are updated so that the distributed system maintains a consistent and unified record of sample location, processing history, and workflow progress.

As a non-limiting, practical example of the foregoing concepts, a clinical testing organization may operate three autonomous laboratory facilities (e.g., Facility A, Facility B, and Facility C), each functioning as an independent node within the distributed hierarchical architecture. A batch of samples may initially be received at Facility A, where the MES uses LIMS data to determine that the pre-extraction and extraction stages should be executed locally due to available work cells and reagent inventory. After these stages complete, the MES may identify that Facility B has greater capacity for post-amplification processing, and the logistics coordination subsystem may autonomously arrange the inter-facility transfer. Upon arrival, Facility B's scheduler may verify the samples and execute the assigned steps using local instruments and multi-temperature storage as needed. Once complete, the MES may again evaluate network-wide availability and may route the subsequent sequencing preparation stage to Facility C, which triggers another automated transfer.

In some aspects, the hierarchical software architecture described herein may further incorporate one or more artificial intelligence or machine learning (AI/ML) modules configured to enhance the efficiency, robustness, and adaptability of the autonomous workflow management system. These AI/ML modules may be trained using, e.g., one or more of historical and/or real-time data published to LIMS and other system data repositories, including sample processing histories, instrument health indications, reagent consumption patterns, workflow timing statistics, instrument or work cell error/failure rates, and inter-work-cell throughput measurements. In operation, the AI/ML modules may continuously or periodically analyze this data to, e.g., identify patterns, predict system behavior, detect emerging bottlenecks, and estimate future resource utilization.

In an aspect, insights derived from these analyses may be provided to the MES, enabling the MES to dynamically adjust workflow process decisions, including sample routing between work cells, prioritization of particular stages, and allocation of samples to work cells or virtualized computer resources based on predicted availability. For instance, if an AI/ML module predicts that a particular activity work cell will experience a processing bottleneck within the next hour based on real-time throughput trends, the MES may proactively reroute incoming samples to an alternate work cell with greater predicted availability. Similarly, if the model identifies that a downstream stage is likely to complete early across several batches, the MES may dynamically reprioritize and advance subsequent workflow stages to maintain optimal system utilization.

In some aspects, the AI/ML module may provide optimization recommendations or decision support to the scheduler(s), allowing the schedulers to, e.g., refine the execution order of steps, anticipate instrument downtime, or balance loads among multiple instruments in a work cell. For example, if the AI/ML module identifies that a centrifuge within a work cell is trending toward decreased performance based on vibration or cycle-time patterns, the scheduler may temporarily shift upcoming steps to alternate instruments to avoid delays. Likewise, if the model predicts that performing a particular step earlier in the sequence will reduce overall queue time, the scheduler may reorder the execution of steps to optimize throughput. In some aspects, the AI/ML modules may also detect anomalies, forecast instrument degradation, or identify patterns indicative of reagent depletion, enabling proactive maintenance or replenishment without interrupting ongoing processing.

In general, any process discussed in this disclosure that is understood to be computer-implementable may be performed by one or more processors of a computer system, such as system environment 100, as described above. A process or process step performed by one or more processors may also be referred to as an operation. The one or more processors may be configured to perform such processes by having access to instructions (e.g., software or computer-readable code) that, when executed by the one or more processors, cause the one or more processors to perform the processes. The instructions may be stored in a memory of the computer server. A processor may be a central processing unit (CPU), a graphics processing unit (GPU), or any suitable types of processing unit.

A computer system, such as system environment 100, may include one or more computing devices. If the one or more processors of the computer system are implemented as a plurality of processors, the plurality of processors may be included in a single computing device or distributed among a plurality of computing devices. If a system environment comprises a plurality of computing devices, the memory of the computer system may include the respective memory of each computing device of the plurality of computing devices.

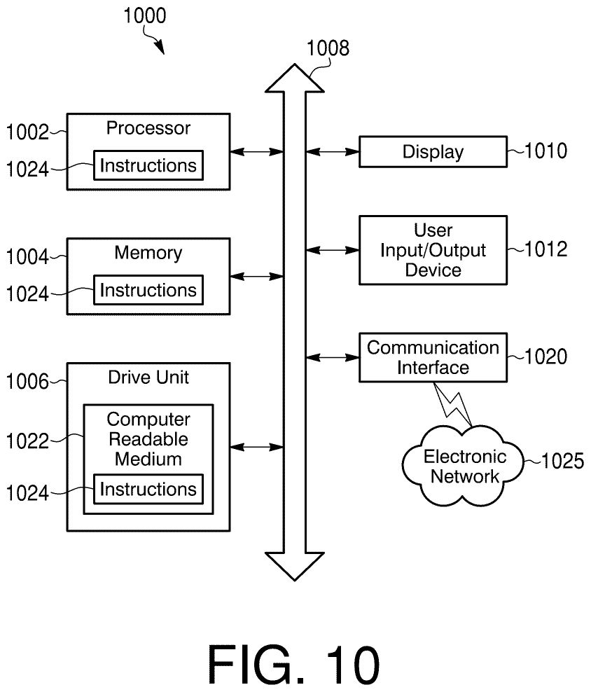

FIG. 10 is a simplified functional block diagram of a computer system 1000 that may be configured as a computing device for executing the processes described herein, according to exemplary embodiments of the present disclosure. FIG. 10 is a simplified functional block diagram of a computer that may be configured according to exemplary embodiments of the present disclosure. In various embodiments, any of the systems herein may be an assembly of hardware including, for example, a data communication interface 1020 for packet data communication. The platform also may include a central processing unit (“CPU”) 1002, in the form of one or more processors, for executing program instructions. The platform may include an internal communication bus 1008, and a storage unit 1006 (such as ROM, HDD, SDD, etc.) that may store data on a computer readable medium 1022, although the system 1000 may receive programming and data via network communications via electronic network 1025 (e.g., voice, video, audio, images, or any other data over the electronic network 1025). The system 1000 may also have a memory 1004 (such as RAM) storing instructions 1024 for executing techniques presented herein, although the instructions 1024 may be stored temporarily or permanently within other modules of system 1000 (e.g., processor 1002 and/or computer readable medium 1022). The system 1000 also may include input and output ports 1012 and/or a display 1010 to connect with input and output devices such as keyboards, mice, touchscreens, monitors, displays, etc. The various system functions may be implemented in a distributed fashion on a number of similar platforms, to distribute the processing load. Alternatively, the systems may be implemented by appropriate programming of one computer hardware platform.

In this disclosure, the term “based on” means “based at least in part on.” The singular forms “a,” “an,” and “the” include plural referents unless the context dictates otherwise. The term “exemplary” is used in the sense of “example” rather than “ideal.” The terms “comprises,” “comprising,” “includes,” “including,” or other variations thereof, are intended to cover a non-exclusive inclusion such that a process, method, or product that comprises a list of elements does not necessarily include only those elements, but may include other elements not expressly listed or inherent to such a process, method, article, or apparatus. Relative terms, such as “about,” “approximately,” “substantially,” and “generally,” are used to indicate a possible variation of ±10% of a stated or understood value. In addition, the term “between” used in describing ranges of values is intended to include the minimum and maximum values described herein. The use of the term “or” in the claims and specification is used to mean “and/or” unless explicitly indicated to refer to alternatives only if the alternatives are mutually exclusive, although the disclosure supports a definition that refers to only alternatives and “and/or.” As used herein “another” may mean at least a second or more.

As used herein, the term “user” generally encompasses any person or entity, such as a laboratory technician, researcher and/or a care provider (e.g., a doctor, etc.), that may desire information, resolution of an issue, or engage in any other type of interaction with a provider of the systems and methods described herein (e.g., via an application interface resident on their electronic device, etc.). The term “electronic application” or “application” may be used interchangeably with other terms like “program,” or the like, and generally encompasses software that is configured to interact with, modify, override, supplement, or operate in conjunction with other software.

Program aspects of the technology may be thought of as “products” or “articles of manufacture” typically in the form of executable code and/or associated data that is carried on or embodied in a type of machine-readable medium. “Storage” type media include any or all of the tangible memory of the computers, processors or the like, or associated modules thereof, such as various semiconductor memories, tape drives, disk drives and the like, which may provide non-transitory storage at any time for the software programming. All or portions of the software may at times be communicated through the Internet or various other telecommunication networks. Such communications, for example, may enable loading of the software from one computer or processor into another, for example, from a management server or host computer of the mobile communication network into the computer platform of a server and/or from a server to the mobile device. Thus, another type of media that may bear the software elements includes optical, electrical and electromagnetic waves, such as used across physical interfaces between local devices, through wired and optical landline networks and over various air-links. The physical elements that carry such waves, such as wired or wireless links, optical links, or the like, also may be considered as media bearing the software. As used herein, unless restricted to non-transitory, tangible “storage” media, terms such as computer or machine “readable medium” refer to any medium that participates in providing instructions to a processor for execution.

Furthermore, while some embodiments described herein include some but not other features included in other embodiments, combinations of features of different embodiments are meant to be within the scope of the invention, and form different embodiments, as would be understood by those skilled in the art. For example, in the following claims, any of the claimed embodiments can be used in any combination.

Thus, while certain embodiments have been described, those skilled in the art will recognize that other and further modifications may be made thereto without departing from the spirit of the invention, and it is intended to claim all such changes and modifications as falling within the scope of the invention. For example, functionality may be added or deleted from the block diagrams and operations may be interchanged among functional blocks. Steps may be added or deleted to methods described within the scope of the present invention.

The above disclosed subject matter is to be considered illustrative, and not restrictive, and the appended claims are intended to cover all such modifications, enhancements, and other implementations, which fall within the true spirit and scope of the present disclosure. Thus, to the maximum extent allowed by law, the scope of the present disclosure is to be determined by the broadest permissible interpretation of the following claims and their equivalents, and shall not be restricted or limited by the foregoing detailed description. While various implementations of the disclosure have been described, it will be apparent to those of ordinary skill in the art that many more implementations are possible within the scope of the disclosure. Accordingly, the disclosure is not to be restricted except in light of the attached claims and their equivalents. This listing of claims will replace all prior versions and listings of claims in the application:

1. A computer system, comprising:
a hierarchical software architecture comprising a plurality of software layers that are collectively configured to automate processing of at least one object through a workflow process, wherein each of the plurality of software layers is configured to be ignorant to a functionality of another of the plurality of software layers, and wherein the plurality of software layers comprise:
a first software layer of the plurality of software layers configured to guide the at least one object between one or more work cells during progression of the at least one object through the workflow process;
one or more second software layers of the plurality of software layers, wherein each of the one or more second software layers are configured to manage execution of one or more steps associated with the workflow process in the one or more work cells; and
one or more third software layers of the plurality of software layers, wherein each of the one or more third software layers are configured to manage execution of tasks, performed by an instrument of a plurality of instruments contained in the one or more work cells, in furtherance of the one or more steps.

2. The computer system of claim 1, wherein the at least one object is a liquid sample.

3. The computer system of claim 2, wherein the liquid sample is a biological sample.

4. The computer system of claim 1, wherein each of the plurality of software layers is configured to publish, during the automated sample processing, system data to a data repository configured to store the system data.

5. The computer system of claim 4, wherein the system data comprises one or more of: progress data associated with the one or more objects, work cell availability data, instrument health data, and material availability data.

6. The computer system of claim 4, wherein each of the plurality of software layers are configured to publish data to the data repository in response to a predetermined event or at a predetermined time interval.

7. The computer system of claim 6, wherein the predetermined event corresponds to completion of an action by any of the plurality of software layers.

8. The computer system of claim 6, wherein the predetermined time interval is a designated time period instituted by a user.

9. The computer system of claim 4, wherein the first software layer is configured to derive decisions to guide the at least one object between the one or more work cells via accessing the system data stored in the data repository.

10. The computer system of claim 1, wherein the first software layer is configured to guide the at least one object between the one or more work cells by controlling operations of a conveyance platform.

11. The computer system of claim 1, wherein the conveyance platform comprises a track along which each of the one or more work cells is positioned.

12. The computer system of claim 1, wherein the first software layer is further configured to manage distribution of one or more materials based at least in part on an inventory level associated with the one or more materials in the one or more work cells.

13. The computer system of claim 12, wherein the one or more materials include: one or more reagents or one or more consumable equipment components.

14. The computer system of claim 1, wherein the first software layer is further configured to generate a unique identifier associated with the at least one object.

15. The computer system of claim 1, wherein the one or more second software layers are configured to receive processing instructions for the at least one object from the first software layer.

16. The computer system of claim 1, wherein the one or more second software layers are configured to communicate updates to the first software layer, the updates including one or more of: object processing notifications, work cell health indications, and inventory availability indications.

17. The computer system of claim 1, wherein each of the one or more second software layers are associated with one of the one or more work cells.

18. The computer system of claim 1, wherein each of the one or more second software layers operably controls one of the one or more work cells

19. The computer system of claim 1, wherein to manage the execution of the one or more steps, each of the one or more second software layers is configured to:
identify which of the one or more third software layers are involved in the execution of the one or more steps; and
transmit step processing instructions to the one or more third software layers identified as being involved in the execution of the one or more steps.

20. The computer system of claim 1, wherein each of the one or more second software layers is configured to monitor a status of one or more instruments and resources within a respective one of the one or more work cells.

21. The computer system of claim 20, wherein each of the one or more second software layers are configured to dynamically reassign responsibility for the execution of the tasks involved in the execution of the one or more steps based on the status of the one or more of the instruments and the resources.

22. The computer system of claim 1, wherein each of the one or more second software layers is configured to simultaneously manage the execution of one or more steps for at least two separate object batches.

23. The computer system of claim 22, wherein each object batch from the at least two separate object batches is at a different point in the workflow process.

24. The computer system of claim 1, wherein each of the one or more third software layers are associated with an instrument of the plurality of instruments contained in the one or more work cells.

25. The computer system of claim 1, wherein the first software layer is further configured to guide the at least one object between a plurality of geographically distributed facilities, each of the plurality of geographically distributed facilities comprising a respective subset of the one or more work cells.

26. The computer system of claim 25, further comprising:
a logistics coordination subsystem configured to coordinate physical transport of the at least one object between the plurality of geographically distributed facilities in furtherance of the workflow process;
wherein the first software layer is further configured to interface with the logistics coordination subsystem to schedule movement of the at least one object between the plurality of geographically distributed facilities.

27. The computer system of claim 1, further comprising:
one or more machine learning modules configured to:
analyze historical data or real-time data associated with the workflow process; and
provide, based on the analysis, one or more insights to the first software layer to enable dynamic adjustment of guidance of the at least one object between the one or more work cells.

28. The computer system of claim 27, wherein the one or more machine learning modules are further configured to:
provide one or more optimization recommendations to at least one of the one or more second software layers to refine execution of the one or more steps associated with the workflow process in the one or more work cells.

29.-54. (canceled)
